(19) United States
(12) Patent Application Publication
(10) Pub. No.: US 2024/0315963 A1
(43) Pub. Date: Sep. 26, 2024

(54) COMPOSITIONS AND METHODS FOR EXTENDED DELIVERY OF API TO TREAT DISEASES OF THE EYE

(71) Applicant: Syed Hasan Askari, Santa Clara, CA (US)

(72) Inventor: Syed Hasan Askari, Santa Clara, CA (US)

(21) Appl. No.: 18/588,184

(22) Filed: Feb. 27, 2024

Related U.S. Application Data
(60) Provisional application No. 63/449,660, filed on Mar. 3, 2023.

(51) Int. Cl. A61K 9/06; A61K 47/32; A61K 47/34

(52) U.S. Cl. CPC A61K 9/06; A61K 47/32; A61K 47/34

(57) ABSTRACT

The present disclosure provides compositions, devices, systems, methods, and kits for treatment of ophthalmic conditions. The compositions and methods of the present disclosure may include a biodegradable polymer containing an API that could be injected directly in the vitreous humor where it forms a gel and acts as a depo and delivers the drug over an extended desired period of time from 1 month to 1 years or more.

### BACKGROUND

Hundreds of millions of people are affected by ophthalmic conditions each year. Ophthalmic conditions include, for example, macular degeneration (e.g., age-related macular degeneration), bulging eyes, cataracts, cytomegalovirus retinitis (CMV retinitis), color blindness, strabismus (crossed eyes), nystagmus, eyestrain, red eyes, diabetic macular edema (DME), diabetic retinopathy, eye floaters and flashes, glaucoma, keratoconus, amblyopia (lazy eye), ocular hypertension, retinal detachment, eyelid twitching, low vision, uveitis, conjunctivitis, presbyopia, dry eyes, excess tearing, blocked tear ducts, corneal disease, chalazion, and hordeolum (sty).

Though some ophthalmic conditions such as eyestrain are minor and/or readily treatable, many ophthalmic conditions may cause significant damage to the eye. For example, age-related macular degeneration (AMD) damages the retina and may cause blindness in one or more eyes. AMD may be “dry” or “wet.” In individuals with dry AMD (also known as non-neovascular or non-exudative AMD), layers of the macula, including photoreceptors and retinal pigment epithelium, atrophy, becoming progressively thinner and less functional. This atrophying may be accompanied by changes in the pigment or color of the macula, as well as the appearance of deposits (drusen) under the retina. Development of pigment discoloration and drusen are common in individuals over 50 years of age. Dry AMD may progress to wet AMD (also known as neovascular or exudative AMD), a condition in which new blood vessels grow in the choroid layer behind the retina (choroidal neovascularization, CNV). Such new vessels are relatively weak and may leak materials such as fluids, lipids, and blood into layers of the retina, including layers of the macula. This leakage may result in the development of scar tissue and reduction or cessation in the functioning of retinal cells.

Over 11 million people in the United States and 196 million people worldwide suffer from dry or wet AMD. The risk of development of AMD increases from about 2% around age 50 to about 30% around age 70. Healthcare costs associated with AMD treatment are estimated to be upwards of $90 billion in North America and $250 billion globally, while costs relating to vision loss are estimate to be upwards of $510 billion in North America and $3 trillion globally. Further, over 2 million people in the United States are affected by glaucoma, and healthcare costs associated with glaucoma in the United States are estimated at upwards of $3 billion.

Existing treatments for ophthalmic conditions have variable effectiveness and often require repeated injections. Some treatments may require 10 or more injections within a given month. Many such treatments carry high risks of side effects including infections and other complications, as well as significant costs. For example, a single treatment course for AMD may cost as much as $20,000. Accordingly, there is a significant need for improved therapies for ophthalmic conditions.

### SUMMARY

The present disclosure provides compositions, methods, and kits for use in treating ophthalmic conditions such as macular degeneration (e.g., age-related macular degeneration, such as wet AMD) and glaucoma. The compositions, methods, and kits provided herein may facilitate a reduction in the number and/or frequency of administrations of a therapeutic composition required to effectively treat an ophthalmic condition, as well as a reduction in the costs and risks associated with treatments of ophthalmic conditions.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows exemplary Elution Studies Using were performed using Jak-12 inhibitor over 6 months:

FIG. 2 shows exemplary Elution Studies Using were performed using Triamcinolone over 5 months; and

FIG. 3 shows exemplary Elution Studies Using using Dasatinib over 4 months.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Definitions

The term “aperture” refers to a hole or opening.

The term “biocompatible” or “biologically compatible,” as used interchangeably herein, refer to materials that are, with any metabolites or degradation products thereof, generally nontoxic to the recipient, and cause no significant adverse effects to the recipient. Generally speaking, biocompatible materials are materials which do not elicit a significant inflammatory or immune response when administered to a patient. In some embodiments a biocompatible material elicits no detectable change in one or more biomarkers indicative of an immune response. In some embodiments, a biocompatible material elicits no greater than a 10% change, no greater than a 20% change, or no greater than a 40% change in one or more biomarkers indicative of an immune response.

The terms “biodegradable”, “bioerodable”, “biologically absorbable,” and “bioresorbable,” in reference to polymers and coatings, are used interchangeably and refer to polymers and coatings that are capable of being completely or substantially completely degraded, dissolved, and/or eroded over time when exposed to bodily fluids such as blood and can be gradually resorbed, absorbed and/or eliminated by the body, or that can be degraded into fragments that can pass through the kidney membrane of a human (e.g., fragments having a molecular weight of about 40,000 Daltons (40 kDa) or less). The process of breaking down and eventual absorption and elimination of the polymer or coating can be caused by, e.g., hydrolysis, metabolic processes, oxidation, enzymatic processes, bulk or surface erosion, and the like. Conversely, a “biostable” polymer or coating refers to a polymer or coating that is not biodegradable.

The term “co-polymer” as used herein refers to a variety of polymer wherein non-identical repeating units are present. A copolymer can be regular or random in the sequence defined by the more than one type of repeating unit. Some types of copolymers are random copolymers, graft copolymers and block copolymers.

The term “effective amount” as used herein with reference to a drug, compound, or pharmaceutical composition is an amount sufficient to effect beneficial or desired results including modulation of clinical manifestations or symptoms such as a decrease in, for example, eye pain, eyelid pain, hazy vision, blurred vision, double vision, seeing flashes of light, seeing bright floating spots, seeing rainbows or halos around lights, seeing floating lines or webs, light or glare sensitivity, vision loss, sudden changes in vision, mid-dilated pupil, oval-shaped pupil, lens specks (glaukomflecken), white areas in the pupil, changes in the color of the iris, swollen eye, eye redness, dry eye, excess tearing, watery eye, eyestrain, sensing blackness spreading over an eye, development of persistent floaters, itchy eye, burning eye, eye discharge, night blindness, amblyopia (lazy eye), strabismus (cross eyes), nystagmus (wandering or jiggling eye), contrast sensitivity, color blindness, presbyopia, nausea, emesis, or high or increased ocular pressure; or increasing the quality of life of those suffering from a disease (e.g., an ophthalmic condition) (for example, increasing physical functioning, decreasing pain, increasing general health, increasing vitality, increasing social functioning), decreasing the dose of other medications, e.g. palliative care medications or other medications, required to treat the disease, delaying the progression of the disease, decreasing time required for resolution of infection and/or symptoms, prolonging vision of patients, and/or prolonging survival of patients. An effective amount can be administered in one or more administrations. In some embodiments, an effective amount of drug, compound, or pharmaceutical composition is an amount sufficient to decrease clinical manifestations of an ophthalmic condition.

The term “inhibit,” “inhibiting,” or “inhibition” as used herein refers to a decrease in activity, response, condition, disease, or other biological parameter. This can include, but is not limited to, the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels. Inhibition of an ophthalmic condition (e.g., AMD) can also include prolonging of vision and/or prevention of blindness, such as prolonging a certain quality of vision (e.g., visual acuity). For example, inhibition an ophthalmic condition can include preserving a certain degree of visual acuity (e.g., as measured upon viewing a Snellen chart), visual field (e.g., as measured by viewing an Amsler grid), contrast or color sensitivity (e.g., as measured by a Pelli Robson test, Farnsworth-Munsell 100 hue test, or Maximum Color Contrast Sensitivity test), drusen spots (e.g., as measured using Fundus photography).

The term “Michael reaction” or “Michael addition” refers to a chemical reaction between a nucleophile (the “Michael donor”) and an activated olefin or alkyne (the “Michael acceptor”) in which the nucleophile adds across a carbon-carbon multiple bond, resulting in a “Michael adduct.” The Michael donor can be a carbon-nucleophile or a hetero-atomic nucleophile.

The term “microorganism” or “microbe” as used herein refers to bacteria, yeast, mold, fungi, protozoa, mycoplasma, as well as viruses (including lipid enveloped RNA and DNA viruses).

The term “modulus” as used herein refers to the ratio of a component of stress or force per unit area applied to a material divided by the strain along an axis of applied force that results from the applied force. The modulus or the stiffness typically is the initial slope of a stress-strain curve at low strain in the linear region. For example, a material has both a tensile and a compressive modulus.

The term “molecular weight” as used herein with reference to a polymer refers to a number average molecular weight, a weight average molecular weight, a peak average molecular weight, a Z average molecular weight, a viscosity average molecular weight, or a melt index of the polymer. The number average molecular weight Mₙ is defined as

$$\frac{\sum {N}_{i} {M}_{i}}{\sum {N}_{i}}$$

where Mᵢ is the molecular weight of a chain and Nᵢ is the number of chains of that molecular weight. The weight average molecular weight M_{w} is defined as

$$\frac{\sum {N}_{i} {M}_{i}^{2}}{\sum {N}_{i} {M}_{i}} .$$

The Z average molar mass M_{z} is defined as

$$\frac{\sum {N}_{i} {M}_{i}^{3}}{\sum {N}_{i} {M}_{i}^{2}} .$$

The viscosity average molecular weight Mᵥ is defined as

$${[ \frac{\sum {N}_{i} {M}_{i}^{1 + a}}{\sum {N}_{i} {M}_{i}} ]}^{1 / a} .$$

The term “patency” or “patent” as used herein refers to the quality or state of being open or unobstructed. It can be expressed in a percentage.

The term “patency maintaining agent” refers to any agent that promotes patency over a suitable period of time. Patency can be measured over a period of days, weeks, months or years.

The term “polymer” as used herein refers to a macromolecule made up of a series of at least about 10, and preferably more, repeating units. In some embodiments, a polymer has at least 10, 20, 30, 40, 50 100, 200, 300, 400, 500, 600, 700, 800, 900, 1000, or more repeating units. Repeating units are linked together by, for example, covalent chemical linkages or non-covalent linkages. In some embodiments, a polymer has a number average molecular weight of more than about 500 g/mol. Polymer types include, but are not limited to, homopolymers (only one type of repeat unit), copolymers (two or more types of repeat units), a blend of homopolymers, a blend of copolymers, and a blend of one or more homopolymers and one or more copolymers.

The term “prevent,” “preventing,” or “prevention” as used herein does not require absolute forestalling of the condition or disease but can also include a reduction in the onset or severity of the disease or condition or inhibition of one or more symptoms of the disease or disorder. Preventing a disease or condition can involve delaying the development of at least one or more symptoms of the disease or condition. For example, preventing a disease or condition can also include prolonging of vision and/or prevention of blindness, such as prolonging a certain quality of vision (e.g., visual acuity), as described herein.

The term “sustained-release composition” refers to a composition that comprises at least one therapeutic agent (i.e., a drug, pro-drug, or co-drug) and which is capable of releasing the therapeutic agent at a relatively steady rate over a prolonged period of time ranging from several days to a week to a month to a year or more, upon administration to an individual in need thereof. For example, a sustained-release composition may provide release of the therapeutic agent over a period of at least 3 days, such as at least 5 days, 7 days, 9 days, 12 days, 14 days, 20 days, 30 days, 45 days, 60 days, 90 days, 135 days, 180 days or longer. In some cases, a sustained-release composition may provide release of the therapeutic agent over a period of at least one week, such as at least one week, two weeks, three weeks, four weeks, one month, two months, three months, four months, five months, six months, or longer.

The term “treating” as used herein refers to means ameliorating, improving or remedying a disease, disorder, condition, or symptom of a disease or condition.

### Polymer Materials

The present disclosure provides compositions, methods, materials, and kits useful for treating conditions of the eye (e.g., ophthalmic conditions). The compositions provided herein comprise a polymer (e.g., a biodegradable polymer) that can be provided to a treatment area (e.g., manually, using an applicator, or by spraying). In some cases, the polymer is applied to the treatment area in a liquid form and gels upon application to the treatment area. In other cases, one or more precursors of the polymer are applied to the treatment area and the polymer is formed at the treatment area (e.g., upon application of a polymerizing agent such as a photoinitiator). In some embodiments, the compositions provided herein include bioabsorbable materials that are absorbed by the body over 2-52 weeks, such as over 13-30 weeks. In some embodiments, the compositions provided herein provide sustained release of a therapeutic agent over several days, such as for at least 3 days, 5 days, 7 days, 10 days, two weeks, three weeks, four weeks, one month, two months, three months, six months, or longer.

Polymers (e.g., biodegradable polymers) of the present disclosure can be prepared using any useful starting materials (e.g., polymerizable materials), and any useful amount or ratio thereof. For example, in some cases, a first material and a second material are used to generate a polymer. In some embodiments, equal amounts of the first and second materials are used to generate the polymer. In other embodiments, a greater amount of the first material is used to generate the polymer. In some embodiments, three or more starting materials (e.g., three or more different chemical entities) are used to generate the polymer. In some embodiments, approximately the same amounts of the three or more starting materials are used to generate the polymer. For example, the first, second, and third starting material are combined in approximately equal amounts to provide the polymer. In other embodiments, a greater amount of the first material is used to generate the polymer, and/or a greater amount of the first and second materials are used to generate the polymer.

In some embodiments, one or more starting materials (e.g., one or more polymeric precursors) are provided to a treatment area and the polymer (e.g., biodegradable polymer) forms at the treatment area (e.g., upon application of a stimulus such as a photoinitiator). In some embodiments, a photoinitiator is a water-soluble photoinitiator that can be initiated by application of light (e.g., visible light). Examples of photoinitiators include, but are not limited to, lithium phenyl-2,4,6-trimethhylbenzoylphosphinate, Irgacure 2959 (1-14-(2-hydroxyethoxy)-phenyl1-2-hydroxy-2-methyl-1-propane-1-one), monoacylphosphineoxide (MAPO) salts (e.g., sodium triphenylphosphine oxide (TPO) and lithium TPO), and bisacylphosphineoxide (BAPO) salts (e.g., BAPO-ONa and BAPO-OLi).

In some cases, one or more starting materials are combined in a vessel that can be used to provide the polymer or the one or more starting materials to a treatment area (e.g., an area of an eye). In some embodiments, one or more starting materials are mixed (e.g., via centrifugation, agitation, stirring, or other mechanical or manual mixing) or otherwise processed upon combination to provide the polymer (e.g., biodegradable polymer). In other cases, one or more starting materials are combined and optionally mixed in a first vessel and then transferred to a second vessel (e.g., for combination with one or more additional starting materials to provide the polymer).

In some embodiments, the polymer (e.g., biodegradable polymer) forms immediately upon combining the starting materials. In some embodiments, combining the starting materials provides a liquid solution, which liquid solution gels over a period of time (e.g., over several seconds, minutes, or hours, and/or upon application to a treatment area). In some embodiments, the liquid solution gels upon initiation of a photoinitiator (e.g., upon application of light such as visible light), polymerizing aid, and/or catalyst, such as at a treatment area. In some embodiments, combining the starting materials provides a liquid solution, which liquid solution gels after introduction of a gelling agent (e.g., prior to application to a treatment area or upon application to a treatment area).

In some embodiments, one or more starting materials are provided in a solution. For example, one or more starting materials are provided in a buffered solution. In some embodiments, a buffered solution is a hydrogen phosphate buffer solution (e.g., a sodium hydrogen phosphate buffer solution). In some embodiments, one or more starting materials are provided in ethanol and/or water, such as a 50/50 water/ethanol solution.

In some embodiments, a polymerizing aid is combined with the starting materials to facilitate generation of the polymer (e.g., biodegradable polymer). In some cases, the polymerizing aid is acetonitrile.

In some embodiments, the polymer is generated under standard temperature and pressure conditions (e.g., ambient temperature and pressure). In some embodiments, the polymer is generated at an elevated temperature (e.g., greater than 25 degrees Celsius, ° C.). In some embodiments, the polymer is generated at a pH between about 6 to about 9, such as between about 6 to about 7, between about 7 to about 8, or between about 8 to about 9. In some embodiments, the polymer is generated at a pH between about 7.5 to about 9, such as between about 7.5 to about 8, between about 8 to about 8.5, between about 8 to about 9, or between about 8.5 to about 9.

In some embodiments, the starting materials comprise one or more materials that are not polymerizable. For example, the starting materials include an agent such as a therapeutic agent (e.g., antioxidant, mineral supplement, vascular endothelial growth factor (VEGF) or VEGF receptor inhibitor, prostaglandin analog, beta blocker, adrenergic agent, miotic (cholinergic), carbonic anhydrase inhibitor, steroid, immunotherapeutic agent, decongestant, coagulating agent, or other agent) or a patency maintaining agent. Details of such agents are provided in a subsequent section. In some embodiments, an agent (e.g., a therapeutic agent) is combined with one or more starting materials prior to formation of the polymer, such as in a vessel that may be used to provide the polymer (e.g., polymer composition comprising the agent) to a treatment area. In other embodiments, an agent (e.g., a therapeutic agent) is added during or after formation of the polymer. For example, in some cases, an agent (e.g., a therapeutic agent) is added to the polymer (e.g., in liquid form) or a solution comprising the polymer prior to provision of the polymer to the treatment area.

In some embodiments, a polymer (e.g., biodegradable polymer) is or comprises a Michael addition polymer, i.e., a polymer formed using a Michael reaction. The Michael donor is a hetero-atomic nucleophile, selected from the group consisting of nitrogen, sulfur, oxygen or phosphorous nucleophiles.

In some embodiments, the Michael donor is a thiol donor and the reaction is a thiol-Michael addition reaction. In some embodiments, the thiol donor is an aromatic thiol. In some embodiments, the aromatic thiol has more than one aromatic ring, i.e., n≥1.

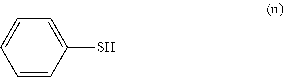

In some embodiments, the Michael donor is a multi-arm donor, such as a compound including multiple aromatic thiol moieties, such as, for example:

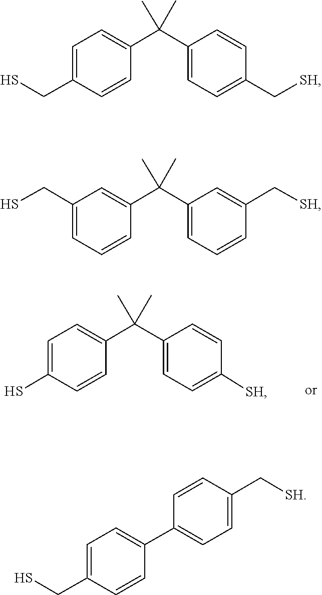

In some embodiments, the Michael donor does not include an aromatic thiol moiety but instead includes one or more non-aromatic thiol moieties. In some embodiments, the Michael donor is a compound including three or more non-aromatic thiol moieties. In some embodiments, the Michael donor is a compound including four or more non-aromatic thiol moieties, such as six non-aromatic thiol moieties or eight non-aromatic thiol moieties. In some embodiments, the Michael donor comprises three or more of the following moieties:

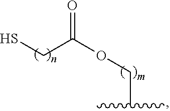

wherein n and m are each independently selected from the group consisting of 1, 2, and 3. In some embodiments, the Michael donor comprises four or more such moieties, such as six such moieties or eight such moieties. In some embodiments, the Michael donor comprises three or more of the following moieties:

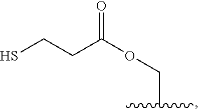

wherein n and m are each independently selected from the group consisting of 1, 2, and 3. In some embodiments, the Michael donor comprises four or more such moieties, such as six such moieties or eight such moieties. In some embodiments, the Michael donor is represented by the formula:

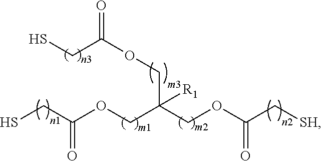

wherein R₁ is selected from the group consisting of H and C₁₋₆ alkyl; n₁, n₂, and n₃ are each independently selected from the group consisting of 1, 2, and 3; and m₁, m₂, and m₃ are each independently selected from the group consisting of 1, 2, and 3. In some embodiments, R₁ is C₁ alkyl. In some embodiments, n₁, n₂, and n₃ are each 2. In some embodiments, m₁, m₂, and m₃ are each 1. In some embodiments, the Michael donor is a compound represented by the formula:

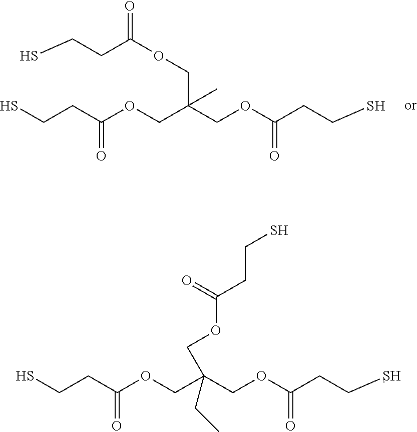 [SMILES: CC(COC(=O)CCS)(COC(=O)CCS)COC(=O)CCS ; CCC(COC(=O)CCS)(COC(=O)CCS)COC(=O)CCS]

In other embodiments, the Michael donor comprises three or more of the following moieties:

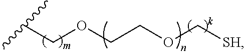

wherein k and m are each independently selected from the group consisting of 1, 2, and 3 and wherein n is selected from the group consisting of 1-500. In some embodiments, the Michael donor comprises four or more such moieties, such as six such moieties or eight such moieties. In some embodiments, the Michael donor comprises three or more of the following moieties:

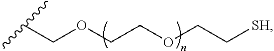

wherein n is selected from the group consisting of 1-500. In some embodiments, the Michael donor comprises four or more such moieties, such as six such moieties or eight such moieties. In some embodiments, the Michael donor is a compound represented by the formula:

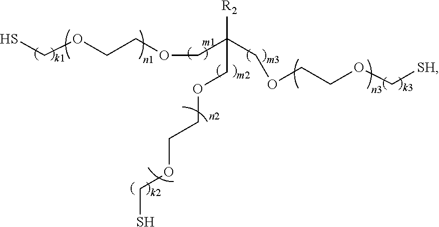

where R₂ is selected from H, C₁₋₆ alkyl, and

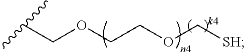

n₁, n₂, n₃, and n₄ (if present) are each independently selected from the group consisting of 1-500, such as 1-20; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. In some embodiments, the Michael donor is a compound represented by the formula:

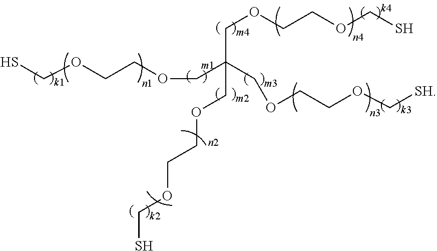

In some embodiments, m₁, m₂, m₃, and m₄ are each 1. In some embodiments, k₁, k₂, k₃, and k₄ are each 2. In some embodiments, the Michael Donor is a compound represented by the formula:

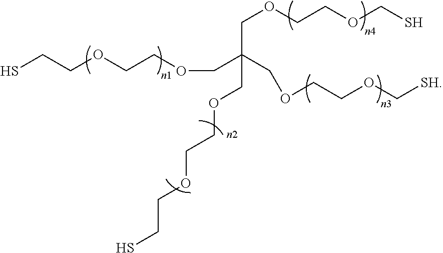

where n₁, n₂, n₃, and n₄ are each independently selected from the group consisting of 1-500. In some embodiments, two or more of n₁, n₂, n₃, and n₄ are the same. In some embodiments, n l , n₂, n₃, and n₄ are all the same.

In some embodiments, the Michael donor does not include a thiol moiety. In some embodiments, a Michael donor includes one or more oxirane moieties. In some embodiments, the Michael donor is a compound represented by the formula:

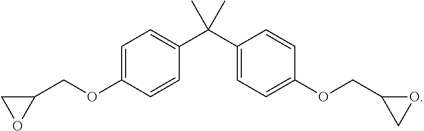 [SMILES: CC(C)(c1ccc(OCC2CO2)cc1)c1ccc(OCC2CO2)cc1]

In some embodiments, the Michael acceptor is an electron-deficient ene selected from the group consisting of acrylates, methacrylates, vinyl sulfones, and maleimides. In some embodiments, the Michael acceptor comprises one or more polyethylene glycol moieties.

In some embodiments, the Michael acceptor is a multi-arm acrylate. In some embodiments, the multi-arm acrylate has greater than 2, greater than 3, greater than four, greater than five, greater than six, or greater than seven arms. In some embodiments, the Michael acceptor includes six acrylate arms. In some embodiments, the Michael acceptor includes eight acrylate arms. Given are 3 structure, 8arm PEG Acrylate, 8-arm methacrylate (both pentaerythritol structure and 8-arm PEG acrylate (hexaglycerol structure)] In some embodiments, an arm of a Michael acceptor is represented by the formula:

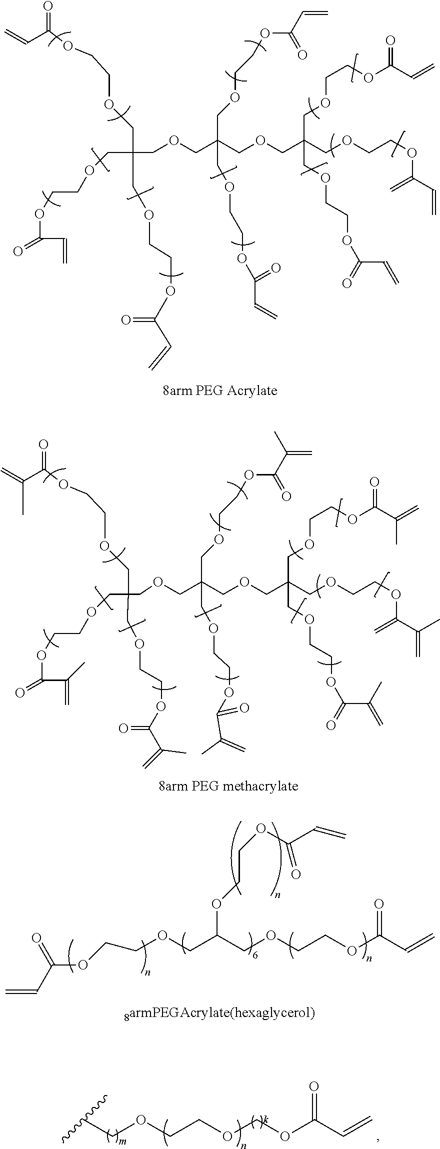

wherein n is selected from the group consisting of 1-500, such as 1-20; and m and k are each independently selected from the group consisting of 1, 2, and 3. In some embodiments, a Michael acceptor includes four such moieties. In some embodiments, a Michael acceptor includes six such moieties. In some embodiments, a Michael acceptor includes eight such moieties. In some embodiments, an arm of a Michael acceptor is represented by the formula:

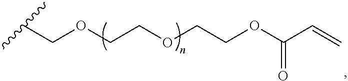

wherein n is selected from the group consisting of 1-500, such as 1-20. In some embodiments, a Michael acceptor includes four such moieties. In some embodiments, a Michael acceptor includes six such moieties. In some embodiments, a Michael acceptor includes eight such moieties. In some embodiments, the Michael acceptor is a compound represented by the formula:

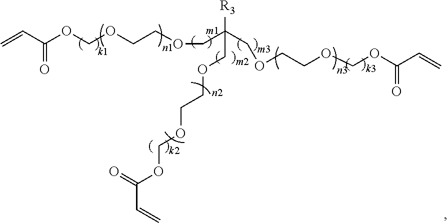

wherein R₃ is selected from the group consisting of H, C₁₋₆ alkyl, and

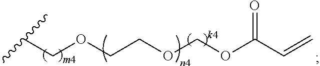

n₁, n₂, n₃, and n₄ (if present) are each independently selected from the group consisting of 1-500, such as 1-20; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. In some embodiments, the Michael acceptor is a compound represented by the formula:

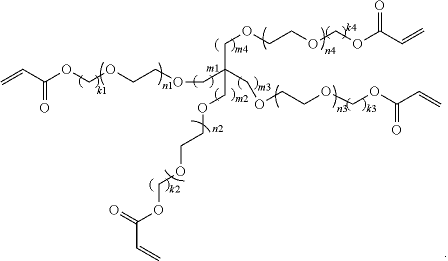

In some embodiments, m₁, m₂, m₃, and m₄ are each 1. In some embodiments, k₁, k₂, k₃, and k₄ are each 2. In some embodiments, the Michael acceptor is a compound represented by the formula:

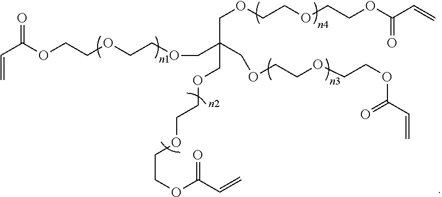

In some embodiments, an arm of a Michael acceptor is represented by the formula

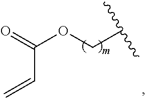

wherein m is selected from the group consisting of 1, 2, and 3. In some embodiments, a Michael acceptor includes four such moieties. In some embodiments, a Michael acceptor includes six such moieties. In some embodiments, a Michael acceptor includes eight such moieties. In some embodiments, the Michael acceptor is a compound represented by the formula:

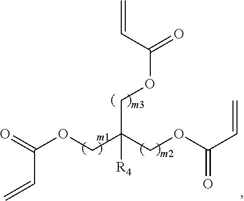

wherein R₄ is selected from the group consisting of H, C₁₋₆ alkyl, and C₁₋₆ hydroxyalkyl; and ml, m₂, and m₃ and are each independently selected from the group consisting of 1, 2, and 3. In some embodiments, R₄ is selected from C₁₋₆ hydroxyalkyl. In some embodiments, R₄ is C₁ hydroxyalkyl. In some embodiments, m₁, m₂, and m₃ and are each 1. In some embodiments, the Michael acceptor is a compound represented by the formula:

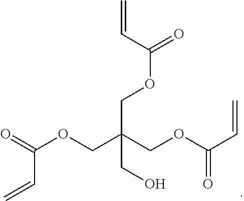 [SMILES: C=CC(=O)OCC(CO)(COC(=O)C=C)COC(=O)C=C]

In some embodiments, the Michael acceptor is a compound represented by the formula:

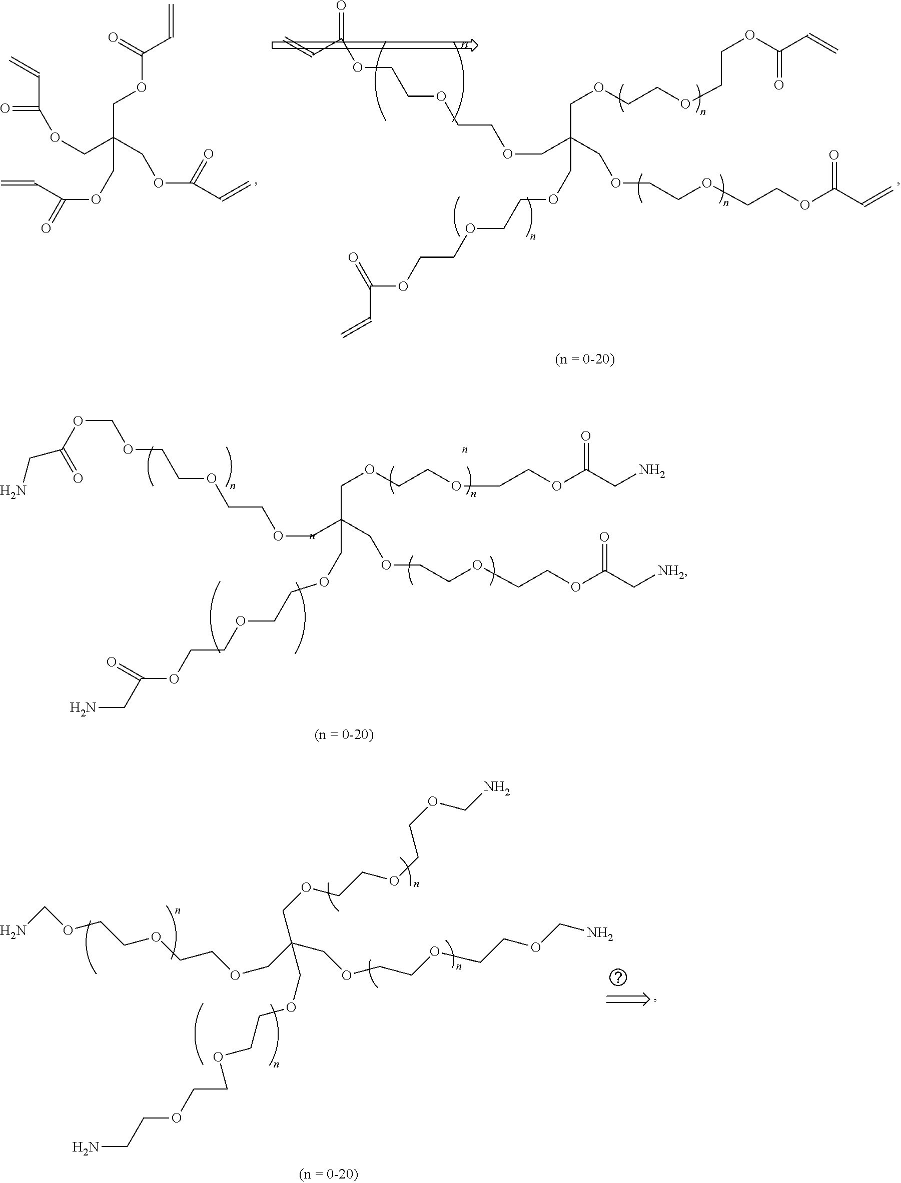

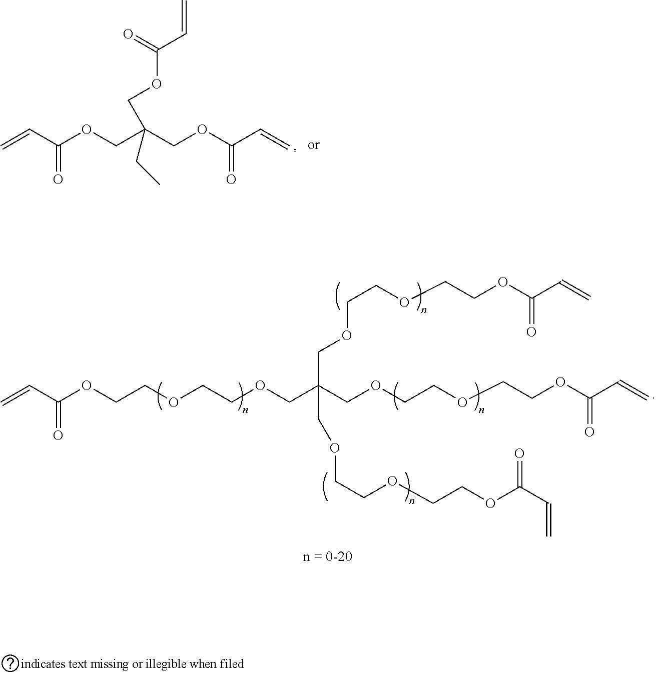

Any useful combination of Michael donors and Michael acceptors can be used in a process of preparing a polymer (e.g., biodegradable polymer) of the present disclosure. In some embodiments, a single Michael donor and a single Michael acceptor are used to prepare the polymer. In some embodiments, a single Michael donor and two or more Michael acceptors are used to prepare the polymer. In some embodiments, two or more Michael donors and a single Michael acceptor are used to prepare the polymer.

In some embodiments, the Michael donor is represented by the formula:

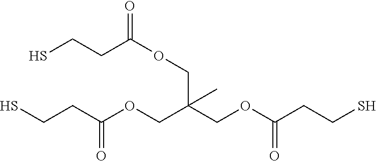 [SMILES: CC(COC(=O)CCS)(COC(=O)CCS)COC(=O)CCS]

and the Michael acceptor is represented by the formula:

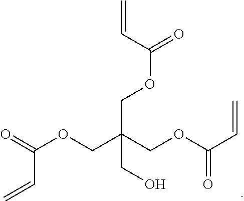 [SMILES: C=CC(=O)OCC(CO)(COC(=O)C=C)COC(=O)C=C]

In some embodiments, the Michael donor is represented by the formula:

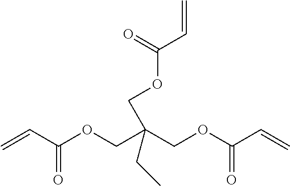 [SMILES: C=CC(=O)OCC(CC)(COC(=O)C=C)COC(=O)C=C]

and the Michael acceptor is represented by the formula:

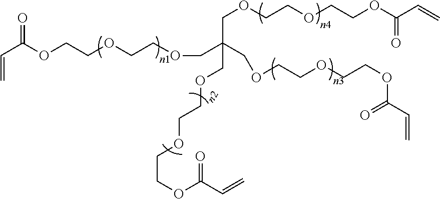

In some embodiments, the Michael donor(s) is represented by the formula:

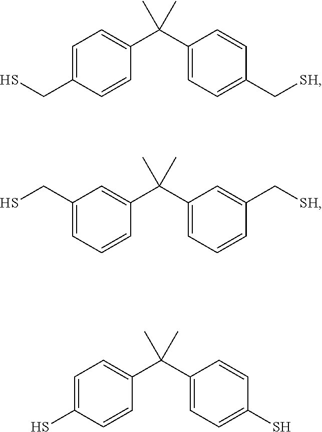 [SMILES: CC(C)(c1ccc(CS)cc1)c1ccc(CS)cc1 ; CC(C)(c1cccc(CS)c1)c1cccc(CS)c1 ; CC(C)(c1ccc(S)cc1)c1ccc(S)cc1]

and/or and the Michael acceptor(s) is represented by the formula:

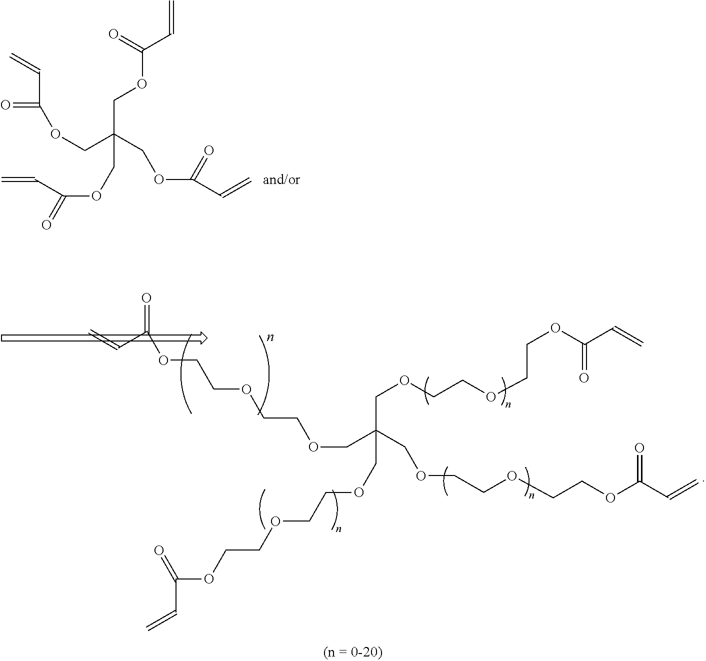

In some embodiments, the Michael donor(s) is represented by the formula:

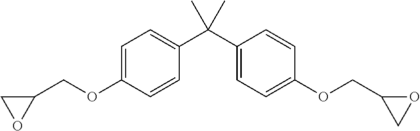 [SMILES: CC(C)(c1ccc(OCC2CO2)cc1)c1ccc(OCC2CO2)cc1]

and the Michael acceptor(s) is represented by the formula:

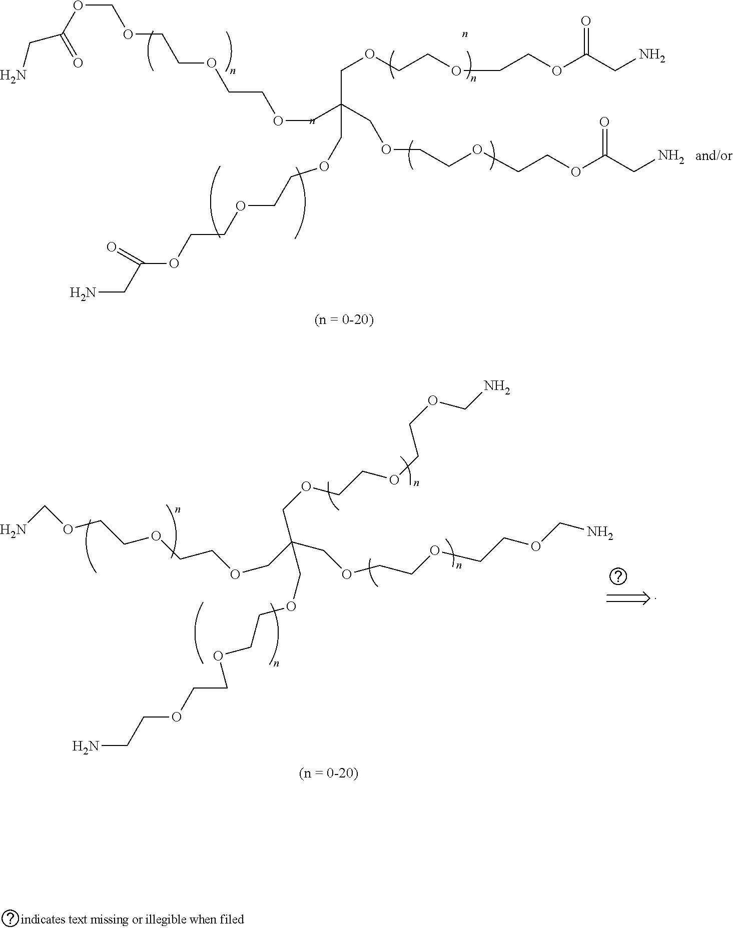

In some embodiments, the Michael donor is represented by the formula:

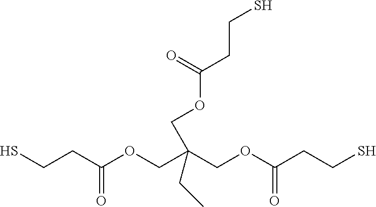 [SMILES: CCC(COC(=O)CCS)(COC(=O)CCS)COC(=O)CCS]

and the Michael acceptors are represented by the formulas:

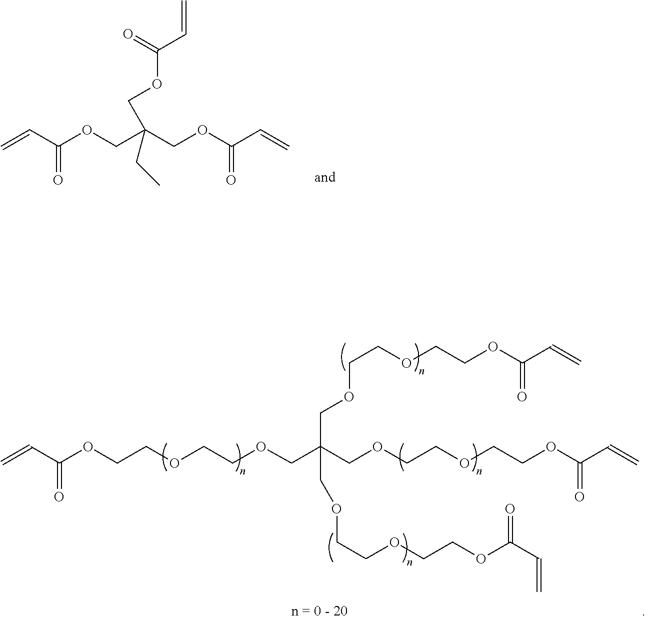

The thiol-Michael addition reaction progresses via an anionic pathway. In some embodiments, the reaction uses a catalyst. Examples of catalysts useful in the reaction include a base, a nucleophile, or a Lewis acid. In alternative embodiments, the reaction is performed without a catalyst using highly polar solvent. The rate limiting step in this reaction is generally the nucleophilic action of the thiolate anion to a vinyl.

In some embodiments, the reaction is base catalyzed (e.g., an amine) to facilitate the reaction between a thiol and an electron-deficient vinyl group to yield a thiolether additional product. The reaction kinetics are dependent on one or more of the following: strength and concentration of the catalyst, the thiol pKa, the steric accessibility of the thiol, nature of the electron withdrawing group coupled to the C═C bond and the polarity and pH of the solvent.

In some embodiments, the reaction uses a photoinitiator (e.g., as described herein). In some cases, the photoinitiator is a water-soluble photoinitiator.

In some embodiments, the polymer is prepared by the reaction of bis-dithiol and a multi-armed acrylate in a Michael reaction. The aromatic ring provides hardness to the material.

In some embodiments, the polymer is prepared by the reaction shown in Scheme 1 below:

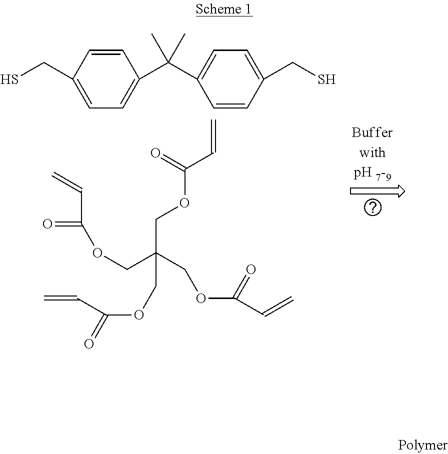

In some embodiments, the polymer is prepared by the reaction shown in Scheme 2 below:

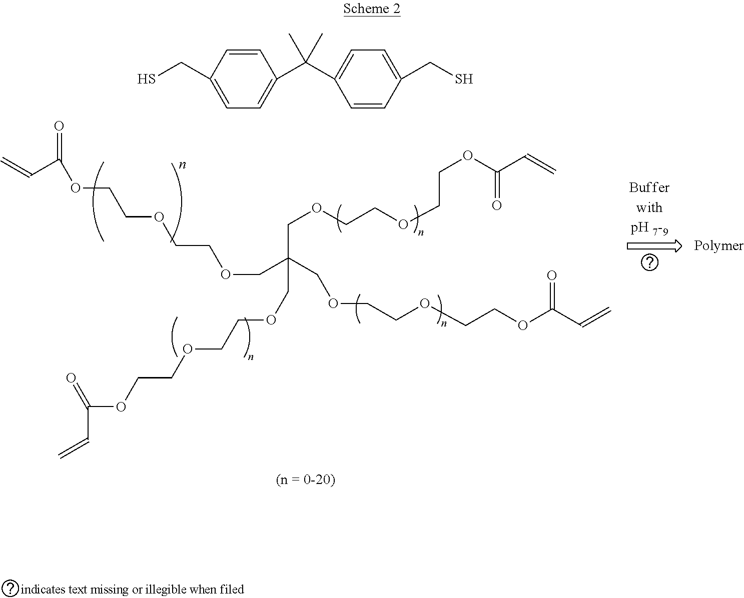

Depending on the modulus of the materials/device desired, the value of n for the PEG unit is adjusted to meet the requirements. For example, if the modulus of the material is desired to be more than 20 megapascal (MPa), such materials are prepared by having PEG chains length of about 5 units, i.e., n=5. If however, however, modulus of less than 1 MPa, then n=10-20. For modulus of 20-1000 MPA, the value of n=0-5. In some embodiments, each n is the same. In other embodiments, one or more n's are different from one another.

In other embodiments, the polymer is prepared by the reaction shown in Scheme 3 below:

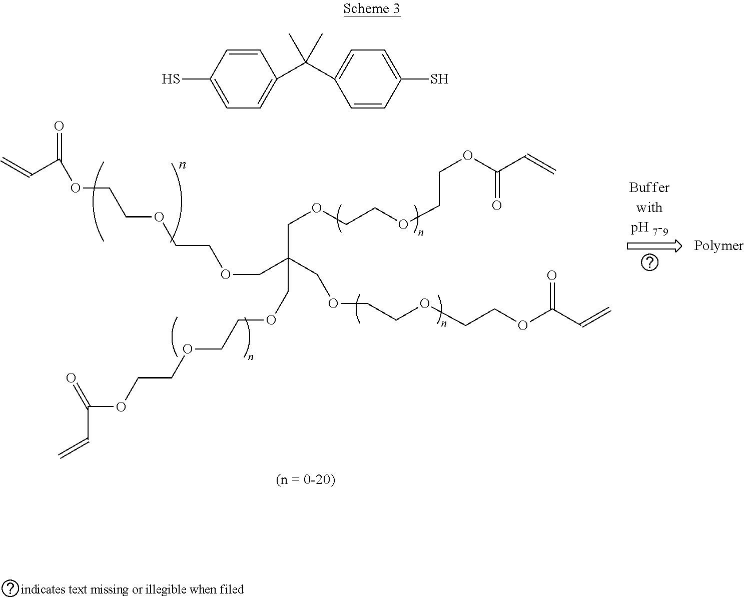

In some embodiments, each n is the same. In other embodiments, one or more n's are different from one another.

In some embodiments, the polymer is prepared by the reaction of bis-gycildyl ether and an amine.

In another embodiment, the polymer is prepared by the reaction shown in Scheme 4 below:

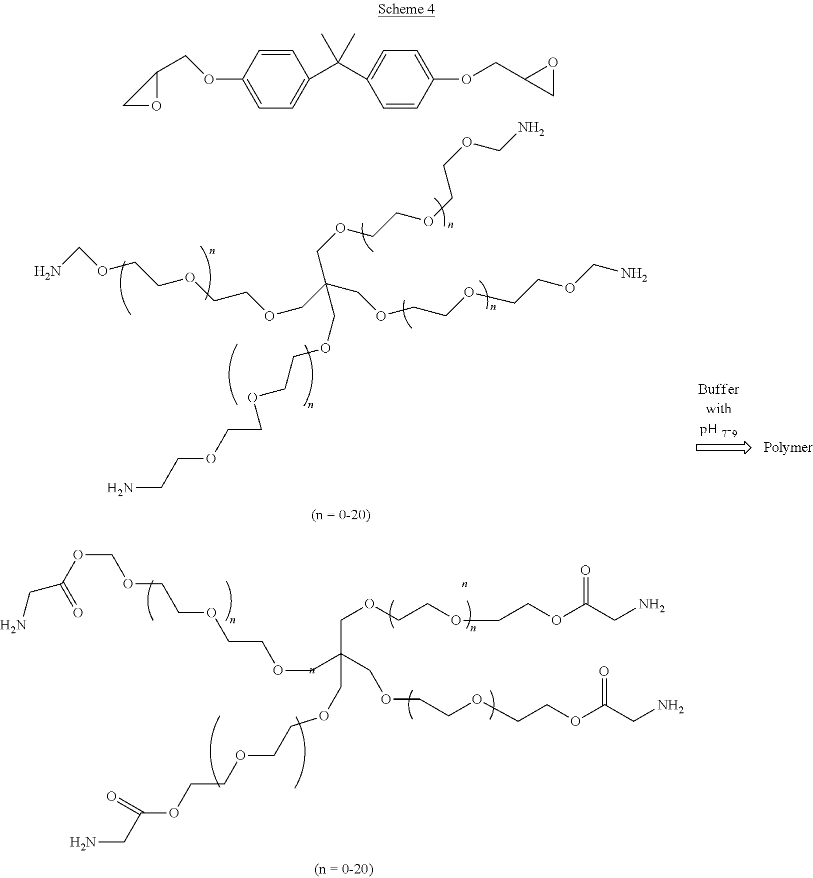

In some embodiments, both an amine containing two ester linkages and a non-ester amine are utilized. By increasing the non-ester containing amine, the degradation time is increased whereas by increasing the ester containing amine, the degradation is decreased. In some embodiments, the polymer (e.g., biodegradable polymer) degrades in between about 2 and about 8 weeks, or more particularly, about 4 and about 8 weeks. In some embodiments, the polymer degrades over one or more month, such as 2 months, 3 months, 4 months, 5 months, 6 months, or longer.

In some embodiments, each n of a given compound used in the reaction is the same. In other embodiments, one or more n's of a given compound used in the reaction are different from one another.

In some embodiments, the polymer is prepared by the reaction of a thiol-containing compound, a compound comprising one or more acrylate moieties, and a compound comprising one or more acrylate moieties and one or more polyethylene glycol moieties, as shown in Scheme 5 below:

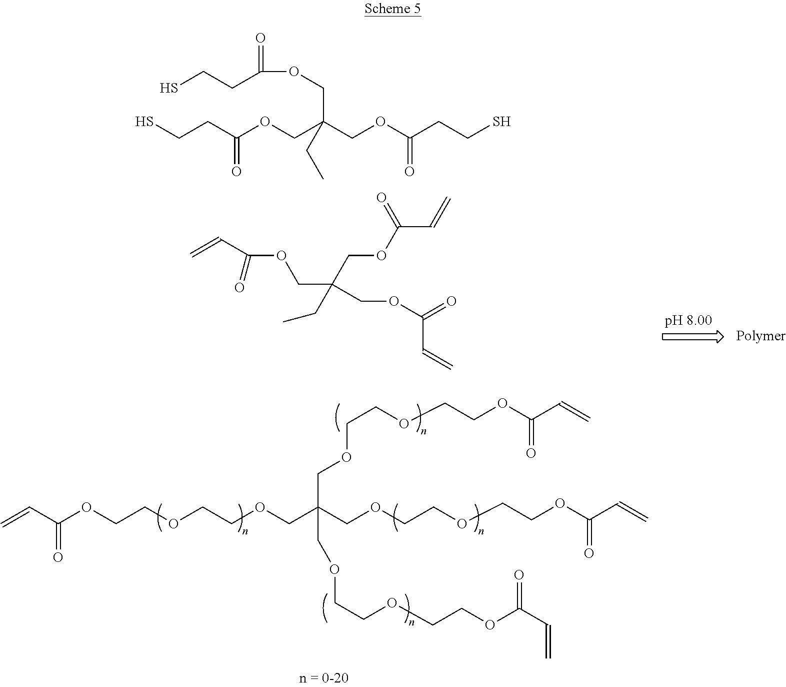

In some embodiments, each n=1-500. In some embodiments, each n=1-20, such as between 1 and 20. In some embodiments, each n is the same. In other embodiments, one or more n's are different from one another.

In some embodiments, the polymer is prepared by the reaction shown in Scheme 6:

In some embodiments, the polymer is prepared by the reaction shown in Scheme 7 below:

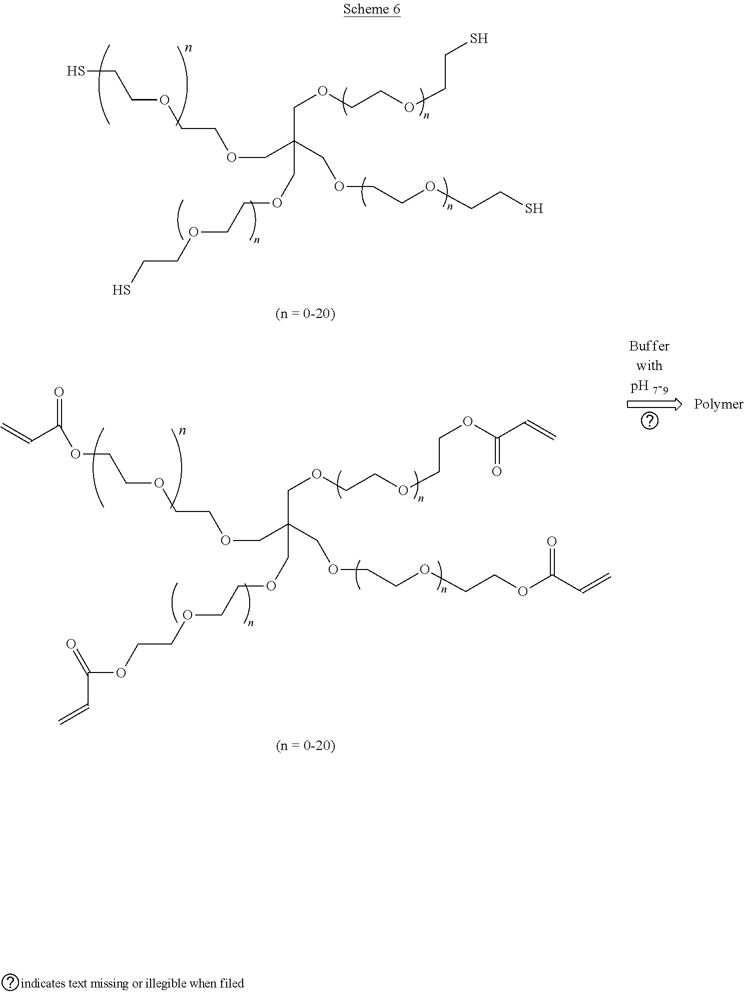

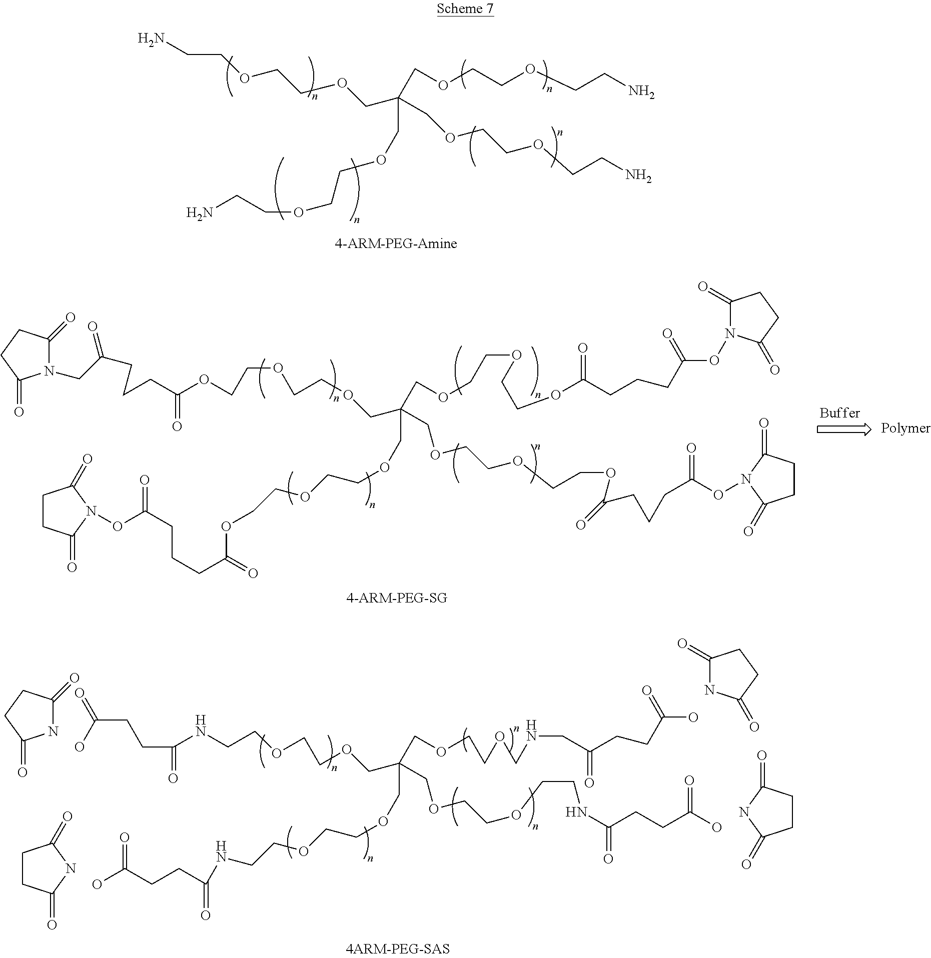

In some embodiments, each n=1-500. In some embodiments, each n=1-20, such as between 1 and 20. In some embodiments, each n is the same. In other embodiments, one or more n's are different from one another

In some embodiments, each n=1-500. In some embodiments, each n=1-20, such as between 1 and 20 (scheme 7). In some embodiments, each n is the same. In other embodiments, one or more n's are different from one another

In some embodiments, the polymer is prepared by the reaction shown in Scheme 8 below:

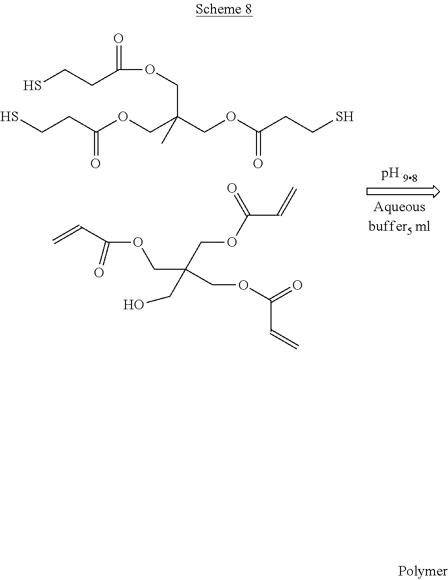

In some embodiments, a polymer (e.g., biodegradable polymer) provided herein is absorbed by the body over 2-8 weeks, such as over 2-4 weeks. In some embodiments, a polymer (e.g., a biodegradable polymer) provided herein is absorbed by the body over one or more months, such as over 2 months, 3 months, 4 months, 5 months, 6 months, or more.

In some embodiments, a polymer (e.g., biodegradable polymer) provided herein includes an agent such as a therapeutic agent. In some embodiments, the agent is included a gel matrix comprising the polymer. In some embodiments, the agent (e.g., therapeutic agent) is dissolved in the polymer (e.g., a gelled or liquid form of the polymer) or a solution comprising the polymer or precursors thereof. In some embodiments, the agent is dispersed (e.g., as particles, such as nanoparticles or microparticles) throughout the polymer (e.g., a gelled or liquid form of the polymer) or a solution comprising the polymer or precursors thereof. In some embodiments, the agent is linked to the polymer or a precursor thereof.

In some embodiments, a polymer (e.g., biodegradable polymer) provided herein is lyophilized or freeze dried. In some embodiments, the polymer is lyophilized or freeze dried in the presence of a cryoprotectant such as dimethyl sulfoxide, ethylene glycol, glycerol, 2-methyl-2,4-pentanediol, propylene glycol, sucrose, or trehalose. In some embodiments, a lyophilized or freeze dried polymer is rehydrated prior to administration or application of the polymer to a treatment site.

### Compositions and Devices

In an aspect, the present disclosure provides a composition comprising a polymer (e.g., a biodegradable polymer) as described herein. In some embodiments, a composition comprises a single polymer (e.g., biodegradable polymer) type. In some embodiments, a composition comprises two or more polymer types, such as two or more polymer types that can interact with one another to form, e.g., a matrix.

In some embodiments, a composition comprises one or more materials in addition to the polymer (e.g., biodegradable polymer). In some embodiments, a composition comprises a solvent such as water and/or ethanol. In some embodiments, a composition comprises a buffered solution such as a sodium hydrogen phosphate buffered solution. In an example, a composition for administration to an eye comprises a solid buffer component. For example, in some cases, a composition comprises a solid buffer component that becomes an aqueous buffer upon addition of a liquid component. In an example, a composition for administration to an eye is buffered to between about 6.8 and 7.5, such as between about 7.0 and about 7.3. In some embodiments, a composition comprises a viscosity enhancer, such as, for example, hydroxyethylcellulose, methylcellulose, hydroxypropylmethylcellulose, polyvinyl alcohol, or polyvinylpyrrolidone.

In another aspect, the present disclosure provides a material or device comprising a polymer (e.g., a biodegradable polymer) as described herein. In some embodiments, the material of the present disclosure comprises a gelled polymer (e.g., biodegradable polymer). In some embodiments, the device of the present disclosure comprises a gelled polymer and an applicator (e.g., tab, syringe, container, or other applicator), removable packaging, or other component. In some embodiments, the size of a device is selected based on the intended patient population (e.g., child vs. adult), the amount of a composition being administered to a patient, and/or the intended treatment area (e.g., area of or around an eye). For example, an applicator (e.g., syringe) comprising a composition for administration of a larger amount of a composition (e.g., a larger dose of a therapeutic agent therein) to an eye (e.g., intravitreal administration) can be a different size than an applicator (e.g., syringe) comprising a composition for administration of a smaller amount of the composition.

In some embodiments, a composition, material, or device comprises an agent such as a therapeutic agent. In some embodiments, a therapeutic agent is selected from the group consisting of an antioxidant, a mineral a supplement, a vascular endothelial growth factor (VEGF) or VEGF receptor inhibitor, a prostaglandin analog, a beta blocker, an adrenergic agent, a miotic (cholinergic), a carbonic anhydrase inhibitor, a corticosteroid, a decongestant, an immunotherapeutic agent, an adrenocorticoid (e.g., corticosteroid or steroid), an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

In some embodiments, a composition, material, or device comprises a therapeutic agent useful in the treatment of a condition such as an ophthalmic condition. In some embodiments, a composition, material, or device comprises a therapeutic agent useful in inhibiting angiogenesis, such as a vascular endothelial growth factor (VEGF) inhibitor or a VEGF receptor inhibitor. Mechanisms for inhibiting angiogenesis (e.g., VEGF-mediated angiogenesis) include, for example, neutralizing antibodies against VEGF or VEGF receptors, small molecule tyrosine kinase inhibitors of VEGF receptors, deploying decoy receptors for VEGF, and using ribozymes that specifically target VEGF mRNA. In some embodiments, a composition, material, or device comprises an agent selected from the group consisting of ranibizumab (Lucentis), bevacizumab (Avastin), prolactin, ramucirumab, ranibizumab, pegaptanib (Macugen), bevasiranib, AGN211745,vatalanib, pazopanib, TG100801, TG101095, AG013958, AL39324, tasquinimod, itraconazole, carboxyamidotriazole, fumagillin analogs (e.g., TNP-470), angiostatin, endostatin, 2-methoxyestradiol, tecogalan, thalidomide, αᵥβ₃ inhibitors, linomide, tetrathiomolybdate, IFN-α, CM101, IL-12 or an analog thereof, platelet factor-4, thrombospondin, suramin, SU5416, VEGF receptor antagonists, VEGF receptor decoy VEGF Trap, angiostatic steroids, heparin, cartilage-derived angiogenesis inhibitory factor, matrix metalloproteinase inhibitor, OPT-302 (Opthea), aflibercept (Eylea), sorafenib (Nexavar), sunitinib (Sutent), pazopanib (Votrient), everolimus (Afinitor), and a combination thereof. In some embodiments, a composition, material, or device comprises an agent selected from the group consisting of pegaptanib (Macugen), ranibizumab, bevacizumab, VEGF receptor decoy VEGF Trap, bevasiranib, AGN211745, sirolimus, vatalanib, pazopanib, TG100801, TG101095, AG013958, AL39324, OPT-302 (Opthea), aflibercept (Eylea), and a combination thereof. In some embodiments, a composition, material, or device comprises an agent selected from vitamin C, vitamin E, beta-carotene, zinc, copper, lutein, zeaxanthin, and a combination thereof. In some embodiments, a composition, material, or device comprises an agent selected from a prostaglandin analog (e.g., latanoprost, bimatoprost, or travoprost), a beta blocker (e.g., timolol or betaxolol), an adrenergic agent (e.g., brimonidine), a miotic (cholinergic) (e.g., pilocarpine), a carbonic anhydrase inhibitor (e.g., dorzolamide, brinzolamide, or acetazolamide), and a combination thereof

In some embodiments, a composition, material, or device comprises a therapeutic agent useful in promoting hemostasis, such as a blood clotting agent or coagulant. In some embodiments, a composition, material, or device comprises an agent selected from the group consisting of von Wiillebrand factor, platelet activating factors, fibrin, fibrinogen, a blood coagulation factor (e.g., FVII (stable factor), FIX (Christmas factor), FX, FXI (plasma thromboplastin), FXII (Hageman factor), FXIII (fibrin stabilizing factor), FVIII, and subtypes thereof), tissue factor, kallikrein, prekallikrein, thrombin, desmopressin, prothrombin complex concentrate, recombinant activated human factor VII, and other agents useful in promoting blood coagulation. In some embodiments, a composition, material, or device comprises an adsorbent chemical such as a zeolite, a hemostatic agent (e.g., antifibrinolytic agent, vitamin K, microfibrillar collagen, chitosan, anhydrous aluminum sulfate, and kaolin), and/or another procoagulant such as tranexamic acid, aminocaproic acid, or aprotinin.

Non-limiting examples of adrenocorticoids for use in compositions, devices, and materials of the present disclosure include betamethasone, cortisone, dexamethasone, hydrocortisone, methylprednisolone, paramethasone, prednisolone, prednisone, and triamcinolone. Non-limiting examples of analgesics for delivery by the device (e.g., pressure equalization tube) of the present disclosure include acetaminophen, aspirin, buprenorphine, butalbital, butorphanol, codeine, dezocine, diflunisal, dihydrocodeine, etodolac, fenoprefen, fentanyl, floctafenine, hydrocodone, hydromorphone, ibuprofen, ketoprofen, ketorolac, levorphanol, magnesium salicylate, meclofenamate, mefenamic acid, meperidine, meprobamate, methadone, methotrimeprazine, morphine, nalbuphine, naproxen, opium, oxycodone, oxymorphone, pentazocine, phenobarbital, propoxyphene, salsalate, and sodium salicylate.

Non-limiting examples of analgesics for use in compositions, devices, and materials of the present disclosure include antipyrine and benzocaine.

Non-limiting examples of anesthetics for use in compositions, devices, and materials of the present disclosure include bupivicaine, tetracaine, procaine, proparacaine, propoxycaine, dimethocaine, cyclomethycaine, chloroprocaine, benzocaine, lidocaine, prilocaln, levobupivicaine, ropivacaine, dibucaine, articaine, carticaine, etidocaine, mepivacaine, piperocaine, and trimecaine.

Non-limiting examples of antibiotics for use in compositions, devices, and materials of the present disclosure include ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In some embodiments, the antibiotic is ciprofloxacin.

Non-limiting examples of anti-fungal drugs for use in compositions, devices, and materials of the present disclosure include amphotericin B, caspofungin, clotrimazole, fluconazole, itraconazole, ketoconazole, voriconazole, terbinafine, and nystatin.

Anti-inflammatory agents for use in compositions, devices, and materials of the present disclosure include both non-steroidal and steroidal anti-inflammatory agents. Representative, non-limiting examples of non-steroidal anti-inflammatory agents include acetic acids, COX-2 inhibitors, fenamates, oxicam derivatives, propionic acids and salicylates. In some embodiments, the acetic acid is selected from the group consisting of diclofenac, etodolac, indomethacin, ketorolac, nabumetone, sulindac, and tolmetin. In some embodiments, the COX-2 inhibitor is celecoxib. In some embodiments, the fenamate is selected from the group consisting of meclofenamate and mefanamic acid. In some embodiments, the oxicam derivative is selected from the group consisting of meloxicam and piroxicam. In some embodiments, the propionic acid is selected from the group consisting of fenoprofen, flurbiprofen, ibuprofen, ketoprofen, naproxen and oxaprozin. In some embodiments, the salicylate is selected from the group consisting of aspirin, diflunisal, magnesium salicylate and salsalate. Representative, non-limiting examples of steroidal active agents dexamethasone, dexamethasone sodium phosphate, hydrocortisone, hydrocortisone acetate, prednisolone, methylprednisolone, prednisone, triamcinolone acetonide, mometasone, budesonide, fluocinolone acetonide, betamethasone, betamethasone sodium phosphate, betamethasone valerate, cortisone acetate, isoflupredone acetate, tixocortol pivalate, triamcinolone alcohol, amcinonide, desonide, fluocinonide, halcinonide, fluocortolone, hydrocortisone-17-butyrate, hydrocortisone-17-valerate, aclometasone dipropionate, betamethasone dipropionate, prednicarbate, clobetasone-17-butyrate, clobetasol-17-propionate, fluocortolone caproate, fluocortolone pivalate, and fluprednidene.

In some embodiments, a composition, device, or material of the present disclosure is configured to deliver at least one antibiotic. In some embodiments, the antibiotic is a penicillin. In some embodiments, the penicillin is amoxicillin. In some embodiments, the antibiotic is a macrolide. In some embodiments, the macrolide is selected from the group consisting of azithromycin, clarithromycin, erythromycin, fidaxomicin or telithromycin. In some embodiments, the antibiotic is a ketolide or a flouroketolide.

In some embodiments, a composition, device, or material of the present disclosure is configured to deliver at least one antibiotic, where the at least one antibiotic is a cephalosporin. In some embodiments, the cephalosporin is a second generation cephalosporin, a third generation cephalosporin, a fourth generation cephalosporin, or a fifth generation cephalosporin. In some embodiments, the second generation cephalosporin is selected from the group consisting of cefaclor, cefonicid, cefprozil, cefuroxime, cefuzonam, cefmetazole, cefotetan, cefoxitin, carbacephems (e.g., loracarbef), and cephamycins (e.g., cefbuperazone). In some embodiments, the third generation cephalosporin is selected from the group consisting of cefcapene, cefdaloxime, cefdinir, cefditoren, cefetamet, cefixime, cefmenoxime, cefodizime, cefotaxime, cefovecin, cefpimizole, cepodoxime, cefteram, ceftamere, ceftibuten, ceftiofur, ceftiolene, ceftizoxime, ceftriazone, cefoperazone, ceftazidime, and oxacephems (e.g., latamoxef). In some embodiments, the fourth generation is selected from the group consisting of cefclidine, cefepime, cefluprenam, cefoselis, cefozopran, cepirome, cefquinome, and oxacephems (e.g., flomoxef). In some embodiments, the fifth generation cephalosporin is selected from the group consisting of ceftobiprole, ceftaroline, and ceftolozane. In some embodiments, the cephalosporin is selected from the group consisting of cefaloram, cefaparole, cefcanel, cefedrolor, cefempidone, cefetrizole, cefivitril, cefmatilen, cemedpidium, cefoxazole, cefrotil, cefsumide, ceftioxide, cefuracetime, and nitrocefin.

In some embodiments, a device of the present disclosure comprises an implantable device. In some embodiments, the device is suitable for administration to and/or placement in or near an eye. For example, in some embodiments, the device is an implantable device (e.g., a drainage implant) made from one or more biodegradable polymers of the present disclosure. In other embodiments, the device is an implantable device coated with one or more biodegradable polymers. In some embodiments, the device (e.g., implantable device) may comprise a therapeutic agent and/or a patency maintaining agent. In some embodiments, a device (e.g., implantable device) does not need to be removed surgically and, in certain embodiments, is drug-eluting. For example, in some embodiments, the device (e.g., implantable device) elutes at least one agent (e.g., a patency maintaining agent) capable of maintaining the patency of a passage or incision for an extended period of time. Alternatively or in addition, the device (e.g., implantable device) elutes one or more therapeutic agents (e.g., as described herein).

The material(s) used to form and/or coat the device (e.g., implantable device) includes one or more biodegradable polymers. The material has a hardness to indentation suitable for withstanding insertion or implantation forces without buckling or bending and, in some cases, for maintaining a passage in a substantially unobstructed, open condition to permit drainage and ventilation of the passage over extended periods of time.

Hardness of a material (e.g., a polymer provided herein or a combination thereof) can be measured by, for example, durometer. Higher numbers indicate harder materials; lower numbers indicate softer materials. There are several scales of durometer used for materials with different properties. The two most common scales are the ASTM D2240 type A and type D scales. The A scale is for softer plastics, while the D scale is for harder ones. Each scale results in a value between 0 and 100, with higher values indicating a harder material.

In some embodiments, a material (e.g., a material comprising one or more biodegradable polymers) has a durometer (hardness) of greater than about 40, greater than about 50, greater than about 60, greater than about 70, greater than about 80, greater than about 85, greater than about 90, greater than about 95, or about 100. In some embodiments, a material has a hardness of between about 80 and about 100, more particularly about 85 and 100, and even more particularly, about 90 and 100. In some embodiments, a device (e.g., implantable device) formed of one or more biodegradable polymers has a hardness of between about 40 and about 100, more particularly about 45 and about 95, or more particularly, about 50 and about 90.

In some embodiments, the material (e.g., biodegradable polymer(s)) is compatible with one or more therapeutic agents (e.g., as described herein). For example, a biodegradable polymer does not and should not destabilize the therapeutic agent or affect its performance.

In some embodiments, the material (e.g., biodegradable polymer(s)) is compatible with an agent for maintaining patency of a passage or aperture (e.g., as described herein).

### Methods of Treating Ophthalmic Conditions

In some embodiments, the polymers (e.g., biodegradable polymers) of the present disclosure and materials and devices comprising the same are useful for treating an ophthalmic condition. In some embodiments, an ophthalmic condition is selected from the group consisting of macular degeneration (e.g., age-related macular degeneration, such as dry AMD or wet AMD), bulging eyes, cataracts, cytomegalovirus retinitis (CMV retinitis), color blindness, strabismus (crossed eyes), nystagmus, eyestrain, red eyes, diabetic macular edema (DME), diabetic retinopathy, eye floaters and flashes, glaucoma, keratoconus, amblyopia (lazy eye), ocular hypertension, retinal detachment, eyelid twitching, low vision, uveitis, conjunctivitis, presbyopia, dry eyes, excess tearing, blocked tear ducts, corneal disease, chalazion, and hordeolum (sty). In some embodiments, an ophthalmic condition is wet AMD. In some embodiments, an ophthalmic condition is glaucoma. In some embodiments, an ophthalmic condition is a primary glaucoma (e.g., primary open-angle glaucoma (also referred to as chronic open-angle glaucoma, chronic simple glaucoma, and glaucoma simplex), primary angle closure glaucoma (also referred to as primary closed-angle glaucoma, narrow-angle glaucoma, pupil-block glaucoma, and acute congestive glaucoma), pigmentary glaucoma, exfoliation glaucoma, or primary juvenile glaucoma. In some embodiments, an ophthalmic condition is developmental glaucoma, secondary glaucoma (e.g., inflammatory glaucoma, phacogenic glaucoma, glaucoma secondary to intraocular hemorrhage, traumatic glaucoma, neovascular glaucoma, drug-induced glaucoma, glaucoma of miscellaneous origin), or absolute glaucoma. In some embodiments, an individual treated with a composition or device of the present disclosure has undergone a laser and/or surgical procedure, such as a photodynamic therapy (e.g., via administration of a drug such as verteporfin and subsequent activation by laser irradiation), laser coagulation therapy, cataract surgery, or surgical procedure to relieve ocular pressure or promote ocular drainage (e.g., by opening a canal via a canaloplasty, by generating a flap and window opening via a trabeculectomy or a nonpenetrating deep sclerectomy, or by implanting a drainage implant).

In some embodiments, a subject treated with a composition or device of the present disclosure has one or more symptoms selected from the group consisting of eye pain, eyelid pain, hazy vision, blurred vision, double vision, seeing flashes of light, seeing bright floating spots, seeing rainbows or halos around lights, seeing floating lines or webs, light or glare sensitivity, vision loss, sudden changes in vision, mid-dilated pupil, oval-shaped pupil, lens specks (glaukomflecken), white areas in the pupil, changes in the color of the iris, swollen eye, eye redness, dry eye, excess tearing, watery eye, eyestrain, sensing blackness spreading over an eye, development of persistent floaters, itchy eye, burning eye, eye discharge, night blindness, amblyopia (lazy eye), strabismus (cross eyes), nystagmus (wandering or jiggling eye), contrast sensitivity, color blindness, presbyopia, nausea, emesis, or high or increased ocular pressure. In some cases, a subject treated with a composition or device of the present disclosure is asymptomatic or does not exhibit a full array of symptoms associated with an ophthalmic condition. In some embodiments, a subject treated with a composition or device of the present disclosure may be known to have had an ophthalmic condition or another condition. In some embodiments, a subject treated with a composition or device of the present disclosure may be suspected of having an ophthalmic condition. In some embodiments, a subject treated with a composition or device of the present disclosure may be undergoing treatment for a condition such as an ophthalmic condition. In some embodiments, a subject treated with a composition or device of the present disclosure may have undergone treatment for a condition such as an ophthalmic condition. In some embodiments, a subject treated with a composition or device of the present disclosure may undergo or have undergone one or more additional treatments for an ophthalmic condition, such as a laser-based procedure and/or a surgical procedure (e.g., as described herein).

In some embodiments, a subject treated with a composition or device of the present disclosure is a human. In other embodiments, a subject treated with a composition or device of the present disclosure is an animal, such as a companion animal (e.g., cat or dog).

In some embodiments, a composition or device for treating a condition (e.g., an ophthalmic condition) comprises an agent useful in the treatment of the condition (e.g., as described herein). In some embodiments, the agent is selected from a patency maintaining agent and a therapeutic agent (e.g., as described herein). In some embodiments, the therapeutic agent is selected from the group consisting of an antioxidant, a mineral supplement, a vascular endothelial growth factor (VEGF) or VEGF receptor inhibitor, a prostaglandin analog, a beta blocker, an adrenergic agent, a miotic (cholinergic), a carbonic anhydrase inhibitor, a corticosteroid, a decongestant, an immunotherapeutic agent, an adrenocorticoid (e.g., corticosteroid or steroid), an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

In some embodiments, a device of the present disclosure comprises a polymer (e.g., biodegradable polymer) of the present disclosure. In some embodiments, a device of the present disclosure comprises a composition comprising a polymer (e.g., biodegradable polymer) of the present disclosure. In some embodiments, the polymer or the composition comprising the polymer is integrated throughout the device. In some embodiments, the polymer or the composition comprising the polymer is localized in one or more regions of the device. In an example, the device is a drainage implant (e.g., for the treatment of glaucoma)and the polymer or the composition comprising the polymer is distributed along a surface of the implant that is configured to contact an area of an eye. In some embodiments, the polymer or the composition comprising the polymer coats all or a portion of the device. In some embodiments, the coating is applied via a spraying or deposition process. In some embodiments, the coating is applied by dipping or submerging the device into a solution comprising the polymer or the composition comprising the polymer, or starting materials useful in providing the same. In some embodiments, the coating is applied after formation of the polymer. In some embodiments, the polymer forms on a surface of the device or within throughout the entire device or a region of the device. In an example, the device is an implantable device (e.g., a drainage implant) that is saturated with a solution comprising a first starting material useful in generating the polymer and a second starting material useful in generating the polymer is subsequently provided to the implantable device to promote formation of the polymer throughout the implantable device. In some embodiments, the device comprises one or more features useful in promoting or localizing polymerization of starting materials useful in generating a polymer of the present disclosure on and/or within the device. For example, in some embodiments, the device comprises one or more features on the surface of the device for promoting formation of the polymer on the surface of the device, such as one or more protrusions or grids.

In general, the effective amount of the patency maintaining agent and/or one or more therapeutic agents, such as one or more agents effective in treating an ophthalmic condition, will be in the range of from about 0.1 μg per gram of treated tissue to about 500 μg per gram of treated tissue. In specific embodiments it may range from about 0.1 μg per gram of treated tissue to about 200 μg per gram of treated tissue, in still other embodiments it may be from about 100 μg per gram of treated tissue to about 500 μg per gram of treated tissue. These amounts are expressed in terms of local effective concentrations within the treated tissue, and it should be understood that the concentrations will range from a relatively high level immediately adjacent to the device (e.g., applied or implanted device) or composition, to insignificant levels in distant tissues.

A device (e.g., implantable device) comprising at least one therapeutic agent will be configured to contain a suitable amount of the at least one therapeutic agent. For example, the device (e.g., implantable device) will contain a suitable amount of the at least one therapeutic agent to deliver a sufficient amount of the at least one therapeutic agent to a patient. For therapeutic agents designed to target a microorganism, the suitable amount of the at least one therapeutic agent will be determined by the microorganism(s) targeted. In some embodiments, the device (e.g., implantable device) contains from about 1,000 μg to about 50,000 μg of the at least one therapeutic agent. In some embodiments, the device (e.g., implantable device) contains between about 1,000 μg to about 5,000 μg, about 5,000 μg to about 10,000 μg, about 1,000 μg to about 2,000 μg, about 2,000 μg to about 3,000 μg, about 3,000 μg to about 4,000 μg, about 4,000 μg to about 5,000 μg or more of the at least one therapeutic agent. In some embodiments, the device (e.g., implantable device) contains between about 1,000 μg and about 2,000 μg, about 200 μg and about 300 μg, about 300 μg and about 400 μg, about 5,000 μg and about 6,000 μg, about 6,000 μg and about 7,000 μg, about 7,000 μg and about 8,000 μg, about 8,000 μg and about 9,000 μg of the at least one therapeutic agent. In some embodiments, the device (e.g., implantable device) contains up to about 200 mg of the at least one therapeutic agent. In some embodiments, the device (e.g., implantable device) contains up to about 100 mg of the at least one therapeutic agent. In some embodiments, the device (e.g., implantable device) contains between about 1 mg to about 200 mg of the at least one therapeutic agent, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., implantable device) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one therapeutic agent. In some embodiments, the device (e.g., implantable device) contains about 25 mg, about 50 mg, about 75 mg, about 100 mg, about 125 mg, about 150 mg, about 175 mg, or about 200 mg of the at least one therapeutic agent. A device (e.g., implantable device) comprising at least one patency maintaining agent will be configured to contain a suitable amount of the at least one patency maintaining agent. For example, the device (e.g., implantable device) will contain a suitable amount of the at least one patency maintaining agent to deliver a sufficient amount of the at least one patency maintaining agent to a patient, and/or to maintain the patency of the device for a proscribed period of time. In some embodiments, the device (e.g., implantable device) contains from about 1,000 μg to about 50,000 μg of the at least one patency maintaining agent. In some embodiments, the device (e.g., implantable device) contains between about 1,000 μg to about 5,000 μg, about 5,000 μg to about 10,000 μg, about 1,000 μg to about 2,000 μg, about 2,000 μg to about 3,000 μg, about 3,000 μg to about 4,000 μg, about 4,000 μg to about 5,000 μg or more of the at least one patency maintaining agent. In some embodiments, the device (e.g., implantable device) contains between about 1,000 μg and about 2,000 μg, about 200 μg and about 300 μg, about 300 μg and about 400 μg, about 5,000 μg and about 6,000 μg, about 6,000 μg and about 7,000 μg, about 7,000 μg and about 8,000 μg, about 8,000 μg and about 9,000 μg of the at least one patency maintaining agent. In some embodiments, the device (e.g., implantable device) contains up to about 200 mg of the at least one patency maintaining agent. In some embodiments, the device (e.g., implantable device) contains up to about 100 mg of the at least one patency maintaining agent. In some embodiments, the device (e.g., implantable device) contains between about 1 mg to about 200 mg of the at least one patency maintaining agent, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., implantable device) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one patency maintaining agent. In some embodiments, the device (e.g., implantable device) contains about 25 mg, about 50 mg, about 75 mg, about 100 mg, about 125 mg, about 150 mg, about 175 mg, or about 200 mg of the at least one patency maintaining agent.

In some embodiments, a device is configured for placement in or on a given region of a body, such as a region of a body that requires treatment. For example, in some embodiments, a device is configured for placement in an area of an eye. In some embodiments, the size and shape of the device are selected based on the area of the eye where the device will be placed. In an example, the size and shape of the device are selected for placement in the back of the eye (e.g., an eye of an adult or an eye of a child). In some embodiments, the size and shape of the device are selected based on the size (e.g., gender and/or age) of an individual that will be treated with the device.

In some embodiments, the composition and/or device (e.g., implantable device) is useful in treating an ophthalmic condition by maintaining patency of an aperture formed in an area of an eye (e.g., to promote fluid drainage) for a certain period of time. In some embodiments, the device elutes or releases a patency maintaining agent but does not elute or release a therapeutic agent.

In some embodiments, the device (e.g., implantable device) elutes or releases a patency maintaining agent and at least one therapeutic agent (e.g., as described elsewhere herein). In some embodiments, the at least one therapeutic agent is selected from the group consisting of an antioxidant, a mineral supplement, a vascular endothelial growth factor (VEGF) or VEGF receptor inhibitor, a prostaglandin analog, a beta blocker, an adrenergic agent, a miotic (cholinergic), a carbonic anhydrase inhibitor, a corticosteroid, a decongestant, an immunotherapeutic agent, an adrenocorticoid, an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

In some embodiments, the composition and/or device (e.g., implantable device) is useful in treating an ophthalmic condition without administration of antibiotics. In some embodiments, such a composition and/or device may elute one or more therapeutic agents other than an antibiotic. For example, in some cases, a composition and/or device may elute one or more therapeutic agents for inhibiting angiogenesis (e.g., as described herein).

Advantageously, the methods of the present disclosure permit treatment of an ophthalmic condition via promotion of fluid drainage in an eye while keeping the drainage passage open.

In some embodiments, a device (e.g., implantable device) is maintained in a body (e.g., in or around an eye) for about 10 minutes, about 20 minutes, about 30 minutes, about 40 minutes, about 50 minutes, about 60 minutes, about 90 minutes, about 120 minutes, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 1.5 days, about 2 days, about 4 days, about 1 week, or longer. In some embodiments, a device (e.g., implantable device) is maintained in a body (e.g., in or around an eye) for longer than about 1 week, such as about 2 weeks, about 3 weeks, about 1 month, or longer. In some embodiments, a device (e.g., implantable device) is maintained in a body (e.g., in or around an eye) until all or a portion of a therapeutic agent (e.g., as described herein) is delivered to a treatment site. In some embodiments, all or a portion of a composition or device (e.g., implantable device) applied to a treatment site dissolves or otherwise changes form over a period of time, such as over several days, weeks, or months.

In some embodiments, a placement guide or tool is used to assist in applying or implanting the device (e.g., drainage implant). In some embodiments, applying the device involves removing an applicator such as a contact paper to provide the device to a treatment site. In another example, the device is an implantable device to which a polymer (e.g., a biodegradable polymer) of the present disclosure is applied, and the polymer adheres the device to the treatment site. In another example, the device is an implantable device that is applied to a treatment site to which a polymer (e.g., a biodegradable polymer) has previously been applied, and the polymer adheres the device to the treatment site.

In some embodiments, a composition comprising a polymer (e.g., biodegradable polymer) of the present disclosure is a solution. In some embodiments, a composition comprising a polymer (e.g., biodegradable polymer) of the present disclosure is or comprises a gel. In some embodiments, a composition is directly applied to a treatment site (e.g., in or around an eye). In an example, the composition is or comprises a gel and the gel is applied directly to the treatment site. In another example, the composition is or comprises a solution and the solution is applied to the treatment site by spraying. In such an example, the polymer hardens and/or solidifies after application of the composition to the treatment site.

### Methods of Manufacture

The present disclosure further provides methods of manufacturing the devices (e.g., implantable devices) described herein. The devices of the present disclosure can be manufactured by any suitable method.

In some embodiments, the device (e.g., implantable device) is manufactured by molding the device (e.g., by injection molding or another method) and then providing a polymer of the present disclosure to the device. In some embodiments, the polymer is formed on or within the device. In other embodiments, the polymer is provided (e.g., applied) to a surface of the device.

### Systems and Kits

The present disclosure further provides kits comprising a composition or device comprising a polymer (e.g., a biodegradable polymer) described herein. In some embodiments, the kit includes a device (e.g., an implantable device) as well as one more components useful in connection with the same.

In some embodiments, the kit comprises a first starting material useful in generating the polymer and a second starting material useful in generating the polymer. In some embodiments, one or more starting materials are provided as solids, which solids are optionally provided with a solid buffer component. In other embodiments, one or more starting materials are provided in solutions, which solutions are optionally buffered solutions. In some embodiments, the first starting material is a Michael acceptor. In some embodiments, the second starting material is a Michael donor. In some embodiments, the kit further comprises a third starting material. In some embodiments, the first starting material and the second starting material are provided in separate containers. In some embodiments, the kit further comprises a catalyst or photoinitator for promoting generation of the polymer. In some embodiments, the first starting material and/or the second starting material are provided in a syringe. In some embodiments, the kit further comprises instructions for preparing the polymer from the first starting material and the second starting material. In some embodiments, such instructions comprise instructions to combine the first starting material with the second starting material and to apply the resultant solution (e.g., a solution comprising the polymer or a precursor to the polymer) to the treatment site within, for example, about 1 minute, about 5 minutes, about 10 minutes, about 15 minutes, about 20 minutes, about 30 minutes, about 45 minutes, about 1 hour, or about 2 hours. In some embodiments, such instructions comprise instructions to apply the resultant polymer or solution comprising the polymer or a precursor thereof to the treatment site by spraying the material on the treatment site. In some embodiments, such instructions comprise instructions to apply the starting materials to the treatment site to generate the polymer at the treatment site.

### EXAMPLES

### Example 1: Preparation of Materials

A stirring solution of trimethylolpropane tris(3-mercaptopropionate) (1 ml) (Scheme 7) in 5 ml sodium hydrogen phosphate buffer solution (pH 9.8) was mixed in a Falcon tube with trimethylolpropane ethoxylate triacrylate (0.75 ml) and heated to 45° C. in a water bath for 24 hours. No reaction was observed. To this solution was added acetonitrile (1 ml), after which a polymer was formed in 30 minutes. The hard and brittle polymer was removed from the Falcon tube. A 0.2 g sample of the polymer was placed in 20 ml buffer at pH 8.00 in a 50° C. oven to study the degradation characteristics of the material. The material did not degrade or dissolve even after 1.5 years under the conditions. Thus the degradation time is more than 1.5 years at pH 8.0.

In another embodiment to this reaction, the solvent was changed to acetonitrile (1 ml) and a polymer was formed within 30 minutes.

### Example 2: Preparation of Materials

A stirring 4-Arm PEG-10K-Acrylate (10K MW, 0.5 gm.) solution in water (0.75 ml) was vortex mixed in a 20 ml Falcon tube with 0.25 ml sodium hydrogen phosphate buffer solution (pH 9.8) at ambient temperature. To this mixture was added 4,4′-bis(mercaptomethyl)biphenyl solution (0.18 g) and the mixture was placed in 45° C. water bath. A polymer was formed in about 2-4 minutes. The polymer is bouncy and absorbs less than 10% water by weight.

Example 3: Preparation of Material and Device

A stirring 4-Arm PEG-Acrylate (10K MW, 0.5 g) solution in water (0.75 ml) was vortex mixed in a 20 ml Falcon tube with 0.25 ml sodium hydrogen phosphate buffer solution (pH 9.80) at ambient temperature. To this mixture was added trimethylolpropane tris(mercapto propionate) solution (0.40 g) after mixing for about 20 seconds, a polymer was formed in about 60-90 seconds at ambient temperature.

### Example 4: Preparation of Material and Device—at Lower pH and With Two Acrylate Monomers

A stirring 4-Arm PEG-Acrylate (10K MW, 0.25 gm) solution in water (0.50 ml) was vortex mixed with 0.25 ml sodium hydrogen phosphate buffer solution (pH 8.0) at ambient temperature in a 20 ml Falcon tube. To this mixture was added trimethylolpropane tris(mercapto propionate (0.08 g) and trimethylol propane ethoxylate triacrylate (0.06 g). The reactive mixture was vortex mixed for about 30 seconds. It takes about 3-4 minutes to form the gel. The degradation time was 20 days at pH 8.00.

### Example 5: Preparation of Material and Device

A stirring 4-Arm PEG-Acrylate (10K MW, 0.25 gm) solution in water (0.50 ml) was vortex mixed with 0.25 ml pH sodium hydrogen phosphate buffer solution (pH 8.0) at ambient temperature. To this mixture was added trimethylolpropane tris(mercapto propionate (0.08 g) and trimethylol propane ethoxylate triacrylate (0.06 g). The reactive mixture was vortex mixed for about 30 seconds and poured into a mold of the device shape desired and allowed to gel inside the mold. It takes about 3-4 minutes to form the gel. After removing the mold, the desired parts are obtained and characterized. In some embodiments, the device shape is a rectangular, ovular, or circular pad.

The mold was placed in the physiological buffer for the degradation study. The degradation time was 20 days.

### Example 6: Preparation of Material and Device With Improved Monomer Solubility—Lower pH

A 4-Arm PEG-Acrylate (10K MW, 0.1 g) powder was mixed with 4 ml sodium hydrogen phosphate buffer solution (0.01 molar, pH 7.4) in a 20 ml Falcon tube at ambient temperature. To this mixture was added 4-arm-PEG-10K-Thiol (0.1 g) and vortex mixed for 2 minutes. It appears that there are some undissolved powders floating. The mixture was placed at ambient temperature to react. The gel formation observed at about 7 minutes when a blob was formed and separated from the liquid. The gel formation continued until to about 15 minutes when the entire liquid gelled.

### Example 7: Preparation of Material and Device—Lyophilized Gel

A 4-Arm PEG-Acrylate (10K MW, 0.2 g) powder was mixed with 8 ml sodium hydrogen phosphate buffer solution (0.01 molar, pH 7.4) in a 20 ml Falcon tube at ambient temperature. To this mixture was added 4-arm-PEG-10K-Thiol (0.2 g) and vortex mixed for 2 minutes. It appears that there are some un-dissolved powders floating. The reactive mixture was poured in 3 steel trays (about 1″×1″) of about equal volumes and allowed to react for about 8 minutes. After this time, the trays were placed at −20° C. in a lyophilyzer for about 20 minutes to freeze completely and then freeze dried for 20 hours under vacuum of 0.01 mTorr and then warmed up to the room temperature. The polymers were removed from the trays. The polymer looks fluffy but does absorb liquid quite quickly (in less than 1-2 seconds on contact with water). The polymer swells but is not bouncy in the dry and wet state. When compressed in dry state, it stays compressed and it takes a long time (1-2 days) for it to get to the original volume.

### Example 8: Preparation of Material and Device—Both Monomers Water Soluble

A 4-Arm PEG-Acrylate (10K MW, 0.1 g) powder was mixed with 4 ml sodium hydrogen phosphate buffer solution (0.01 molar, pH 9.8) in a 20 ml Falcon tube at ambient temperature. To this mixture was added 4-arm-PEG-10K-Thiol (0.1 g) and vortex mixed for 30 seconds. It appears that there are still some undissolved powders floating. The mixture was placed at ambient temperature to react to form the gel. The gel formation observed at about 50-70 seconds with signs of strings formation and separation of the polymer from the remaining liquids. The polymer formation is not uniform and homogeneous. It is likely due to the poor solubility of the 4-Arm PEG-10K Acrylate in the aqueous solution.

### Example 9: Evaluation of Bonding Ability of the Polymer with Beef Steak

Powder mixtures of 4-Arm PEG-Acrylate (10K MW, 0.1 g) and 4-arm-PEG-10K-Thiol (0.1 g) were paced inside a 10 ml syringe. In another syringe 4 ml sodium hydrogen phosphate buffer solution (0.01 molar, pH 9.8) was measured and placed. The front ends of the two syringes were connected with a Luer lock connector and the contents were mixed by pressing the syringe plungers back and forth for about 20 seconds. The reactive mixture was carefully dispensed over a selected area of a fresh beef steak (to simulate an open wound) and the gel formation was observed. It took about 50-70 seconds to form the gel over the steak surface.

The bonding of the polymer to the steak surface was evaluated by careful attempts to remove the polymer from the steak surface. The polymer peeled of as a film from the steak surface. Apparently the polymer did not bond well.

### Example 10: Evaluation of Bonding Ability of the Polymer With Beef Steak and a Longer Gel Time Polymer (Lower pH, 7.86)

Powder mixtures of 4-Arm PEG-acrylate (10K MW, 0.1 g) and 4-arm-PEG-10K-thiol (0.1 g) were placed inside a 10 ml syringe. In another syringe 4 ml sodium hydrogen phosphate buffer solution (0.01 molar, pH 7.86) was measured and placed. The front ends of the two syringes were connected with a Luer lock connector and the contents were mixed by pressing the syringe plungers back and forth for about 20 seconds. The reactive mixture was carefully dispensed over a selected area of the fresh beef steak and the gel formation was observed. It took about 5-6 minutes to form the gel over the steak surface.

The bonding of the polymer to the steak surface was evaluated by careful attempts to remove the polymer from the steak surface. The bonding was not strong, perhaps the polymer was non homogenous and the bonding was observed at some places but peeled off from other locations.

### Example 11: Evaluation of Bonding Ability of the Polymer With Beef Steak and a Longer Gel Time Polymer (Lower pH 7.86, Higher Concentration)

Powder mixtures of 4-Arm PEG-acrylate (10K MW, 0.1 g) and 4-arm-PEG-10K-thiol (0.1 g) were placed inside a 10 ml syringe. In another syringe 2 ml sodium hydrogen phosphate buffer solution (0.01 molar, pH 7.86) was measured and placed. The front ends of the two syringes were connected with a Luer lock connector and the contents were mixed by pressing the syringe plungers back and forth for about 20 seconds. The reactive mixture was carefully dispensed over a selected area of the fresh beef steak and the gel formation was observed. It took about 5-6 minutes to form the gel over the steak surface.

The bonding of the polymer to the steak surface was evaluated by careful attempts to remove the polymer from the steak surface. The bonding was strong and uniform across the entire surface area of the steak and the polymer did not peel off from the surface. During further attempted removal, the polymer broke but did not peel off.

The degradation time of the polymer at 43° C. oven temperature was 11 days at pH 7.86

### Example 12: Longer Gel Time Polymer (Higher Concentration, Lower pH 7.6), 4-arm-PEG20K-acrylate

Powder mixtures of 4-Arm PEG-20K-acrylate_(0.2 g) and 4-arm-PEG-10K-thiol (0.1 g) were placed inside a 10 ml syringe (Scheme 6). In another syringe 2.4 ml sodium hydrogen phosphate buffer solution (0.01 molar, pH 7.6) was measured and placed. The front ends of the two syringes were connected with a Luer lock connector and the contents were mixed by pressing the syringe plungers back and forth for about 20 seconds. The reactive mixture was carefully dispensed in a weighing boat. It took about 16 minutes to form the gel. The gel is bouncy and firm. The degradation time of the polymer at 46° C. oven temperature was 7 days at pH 7.86.

The degradation times and of selected formulations at various pH's are shown in the Table below.

TABLE 1
Details of polymer formulations.

| | | Gel | Degra- | Water | Degra- | |
|---|---|---|---|---|---|---|
| | | Time | dation | bath | dation | |
| | Polymer | (min) | Buffer pH | Temp | time | |
| 1 | SA-01-42 | 16.0 min | 7.86 | 46° C. | 5-7 | days |
| | 4-Arm PEG-20K | | | | | |
| | acrylate and 4-arm- | | | | | |
| | PEG-10K-thiol | | | | | |
| 2 | SA-01-43-A | 6.46 min | 7.6 | 37° C. | 14 | days |
| | 4-Arm PEG-10K | | | | | |
| | acrylate and 4-arm- | | | | | |
| | PEG-20K-thiol | | | | | |
| 3 | SA-01-43-B | 6.46 min | 7.6 | 47° C. | 7 | days |
| | 4-Arm PEG-20K | | | | | |
| | acrylate and 4-arm- | | | | | |
| | PEG-10K-thiol | | | | | |
| 4 | SA-01-43 | 5.40 min | 7.6 | 37° C. | 20 | days |
| | 4-Arm PEG-10K | | | | | |
| | acrylate and 4-arm- | | | | | |
| | PEG-10K-thiol (This | | | | | |
| | polymer had a green | | | | | |
| | dye added to it) | | | | | |

### Example 13: Evaluation of Bonding Ability of the Polymer in Mouse Abdominal Model

Powder mixtures of 4-Arm PEG-10K acrylate (0.1 g) and 4-arm-PEG-10K-thiol (0.1 g) were placed in a 10 ml syringe. In another syringe 2 ml sodium hydrogen phosphate buffer solution (0.01 molar, pH 7.86) was measured and placed. The two syringes were connected with a Luer lock connector and the contents were mixed by pressing the plungers back and forth for about 20 seconds. The reactive mixture was carefully dispensed over a surgically cut and exposed mouse abdomen and skin and the gel formation was observed. It took about 5-6 minutes to form the gel over the organs and the skin surface. The bonding of the polymer to the organs surface was evaluated by careful attempts to remove the polymer from the surface. The bonding was strong and uniform across the entire surface area and the polymer did not peel off from the surface. During further attempted removal, the polymer broke but did not peel off.

### Example 14: Evaluation of Bonding Ability of the Polymer in Pig Mucosa Model

Powder mixtures of 4-Arm PEG-20K acrylate (0.06 g) and 4-arm-PEG-10K-thiol (0.03 g) were placed in a 10 ml syringe. In another syringe 0.6 ml sodium hydrogen phosphate buffer solution (0.01 molar, pH 7.86) containing a small amount of green food dye (for visualization purposes) was measured and placed. The two syringes were connected with a Luer lock connector and the contents were mixed by pressing the plungers back and forth for about 20 seconds. The reactive mixture was carefully dispensed over freshly obtained pig nostrils. It took about 5-6 minutes to form the gel over the pig nostrils mucosa. The bonding of the polymer to the pig nostril was evaluated by careful attempts to remove the polymer from the surface. The bonding was strong and uniform across the entire surface area and the polymer did not peel off from the surface. During further attempted removal, the polymer broke but did not peel off.

### Example 15: Gel Formation Using Soluble 8-Arm-PEG20k Acrylate

Powder mixtures of 8-Arm PEG-20K acrylate (0.12 g) and 4-arm-PEG-10K-thiol (0.03 g) were placed in a 10 ml syringe. In another syringe 2.0 ml sodium hydrogen phosphate buffer solution (0.01 molar, pH 7.86) was measured and placed. The two syringes were connected with a Luer lock connector and the contents were mixed by pressing the plungers back and forth for about 20 seconds. The final contents were pushed into one syringe and the gel time was measured. The gel time was ˜18 min.

The polymer degradation time at 7.86 pH/48° C. was 5 days and at 7.6 pH/48° C. the degradation time was 7 days.

### Example 16: Gel Formation Using Soluble 4-Arm-PEG20k Acrylate

Powder mixtures of 4-Arm PEG-20K acrylate (0.06 g) and 4-arm-PEG-10K-thiol (0.03 g) were placed in a 10 ml syringe. In another syringe 0.6 ml sodium hydrogen phosphate buffer solution (0.01 molar, pH 7.86) was measured and placed. The two syringes were connected with a Luer lock connector and the contents were mixed by pressing the plungers back and forth for about 20 seconds. The final contents were pushed into one syringe and the gel time was measured. The gel time was ˜8 min.

### Example 17: Gel Time Reduction at a Constant pH

Powder mixtures of 4-Arm PEG-20K acrylate (0.05 g) and 4-arm-PEG-10K-thiol (0.025 g) were placed in a 10 ml syringe. In another syringe 0.6 ml sodium hydrogen phosphate buffer solution (0.01 molar, pH 7.86, containing 0.5% by weight methyl cellulose, from Sigma Aldrich, M0512, viscosity 4000 cP for 2% in DI) was measured and placed. The two syringes were connected with a Luer lock connector and the contents were mixed by pressing the plungers back and forth for about 20 seconds. The final contents were pushed into one syringe and the gel time was measured. The gel time was ˜9 min.

### Example 18: Gel Time Reduction at a Constant pH

Powder mixtures of 4-Arm PEG-20K acrylate (0.05 g) and 4-arm-PEG-10K-thiol (0.025 g) were placed in a 10 ml syringe. In another syringe 0.6 ml sodium hydrogen phosphate buffer solution (0.01 molar, pH 7.86, containing 0.25% by weight hydroxyl propyl cellulose, from Sigma Aldrich, 435007, average M_{W} 80,000, Mn 10,000) was measured and placed. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for degradation study. The degradation time was 8 days at pH 7.86 at 47° C. and was 14 days at pH 37° C. The degradation time of the polymer at 37° C. in pH 7.4 is 24 days.

| | | | % Ratio of | | | Degrdation |
|---|---|---|---|---|---|---|
| Formulation | Reactant 1 (SAS) | Reactant 2 (SG) | Reactant 1 &2 | Amine | API | time at pH 7.4 |
| | 4arm-PEG-SAS | 4arm-PEG-SG 10K | SAS/SG Ratio | 4arm-PEG- | N/A | |
| | 10K | | | NH2 10K | | |
| SA-12-19-A | 4arm-PEG-SAS | 4arm-PEG-SG 10K | 50/50 | 4arm-PEG- | N/A | 240 |
| | 10K | | | NH2 5K | | |
| SA-12-19C | 4arm-PEG-SAS | 4arm-PEG-SG 10K | 25/75 | 4arm-PEG- | N/A | 30 |
| | 10K | | | NH2 5K | | |
| SA-12-20A | 4arm-PEG-SAS | 4arm-PEG-SG 10K | 0/100 | Trilysine | N/A | 25 |
| | 10K | | | | | |
| SA-12-23A | 4arm-PEG-SAS | 4arm-PEG-SG 10K | 50/50 | 4arm-PEG- | N/A | 180 |
| | 10K | | | NH2 10K | | |
| SA-12-23B | 4arm-PEG-SAS | 4arm-PEG-SG 10K | 75/25 | 4arm-PEG- | N/A | 224 |
| | 10K | | | NH2 10K | | |
| SA-12-23C | 4arm-PEG-SAS | 4arm-PEG-SG 10K | 25/75 | 4arm-PEG- | N/A | 29 |
| | 10K | | | NH2 10K | | |
| SA-12-23D | 4arm-PEG-SAS | 4arm-PEG-SG 10K | 0/100 | 4arm-PEG- | N/A | 20 |
| | 10K | | | NH2 10K | | |
| SA-12-45 | 4arm-PEG-SAS | 4arm-PEG-SG 10K | 60/40 | 4arm-PEG- | N/A | 120 |
| | 10K | | | NH2 10K | | |
| VV-01 | 4arm-PEG-SAS | 4arm-PEG-SG 10K | 50/50 | 4arm-PEG- | N/A | 180 |
| | 10K | | | NH2 10K | | |
| VV-04 | 4arm-PEG-SAS | 4arm-PEG-SG 10K | 50/50 | 4arm-PEG- | N/A | 210 |
| | 20K | | | NH2 20K | | |

### Example 19: Degradation of Measurements of the Polymer

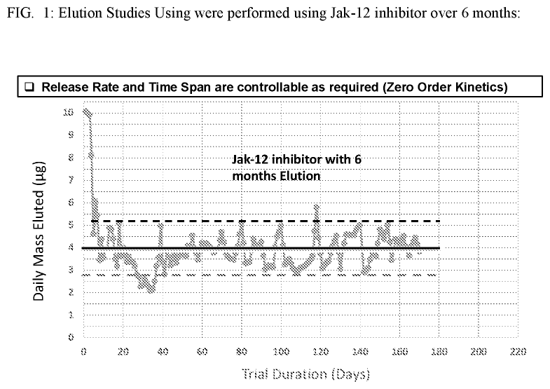

A piece of the polymer sample (about 0.5 g) was placed in a 20 ml buffered saline at 37° C. water bath. The presence of the polymer was observed over time. The degradation time till the total dissolution of the polymer is measured. The degradation time is found to be 2-8 weeks depending on the formulations used. FIG. 1 shows degradation of a polymer over 20 days.

### Example 20: Degradation Time and Drug Elution

A mold piece manufactured per example 5 is placed in a 2 ml buffered saline at 37° C. water bath. The entire 2 ml saline is removed daily from the sample and a new 2 ml saline is added. The process is repeated daily till the device is completely dissolved. The saline sample with drug is analyzed by HPLC, GC to establish the elution rate.

Example 21. Powder mixtures of 4-Arm PEG-10K-SG (12.5 mg) and 4-arm-PEG-10K-SAS (12.5±0.5 mg), 4-arm PEG-20K-NH 2 (25±0.5 mg) were placed in a 10 ml syringe. In another syringe 0.6 ml sodium hydrogen phosphate buffer solution (0.01 molar, pH 6.9. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for degradation study. The degradation time was 200 days at pH 7.40 at pH 37° C.

Example 22. Powder mixtures of 4-Arm PEG-10K-SG (12.5 mg) and 4-arm-PEG-10K-SAS (12.5±0.5 mg), 4-arm PEG-20K-NH 2 (25±0.5 mg) were placed in a 10 ml syringe. To this powder was added 50 mg corticosteroid TMC. In another syringe 0.6 ml sodium hydrogen phosphate buffer solution (0.01 molar, pH 6.9. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for elution study at 37° C. for about 6 months. 2m₁ of the buffer was removed each day and replaced with fresh 7.4 buffer. The amount of eluted product was analyzed by HPLC. The results indicated that about 54 micro grams of the TMC eluted each day.

Example 23. Powder mixtures of 4-Arm PEG-10K-SG (12.5 mg) and 4-arm-PEG-10K-SAS (12.5±0.5 mg), 4-arm PEG-20K-NH 2 (25±0.5 mg) were placed in a 10 ml syringe. To this powder was added 15 mg DST. In another syringe 0.6 ml sodium hydrogen phosphate buffer solution (0.01 molar, pH 6.9. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for elution study at 37° C. for about 6 months. 2m₁ of the buffer was removed each day and replaced with fresh 7.4 buffer. The amount of eluted product was analyzed by HPLC. The results indicated that about 4-6 micro grams of the DST eluted each day

Example 24. Powder mixtures of 4-Arm PEG-10K-SG (12.5 mg) and 4-arm-PEG-10K-SAS (12.5±0.5 mg), 4-arm PEG-20K-NH 2 (25±0.5 mg) were placed in a 10 ml syringe. To this powder was added 15 mg VVN-471. In another syringe 0.6 ml sodium hydrogen phosphate buffer solution (0.01 molar, pH 6.9. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for elution study at 37° C. for about 6 months. 2 ml of the buffer was removed each day and replaced with fresh 7.4 buffer. The amount of eluted product was analyzed by HPLC. The results indicated that about 4-6 micro grams of the VVN471 eluted each day At the end of the term, all of the VVN-471 eluted and the samples dissolved on day 210.

Example 25. Powder mixtures of 4-arm-PEG-10K-SAS (25.5±0.5 mg), 4-arm PEG-20K-NH 2 (25±0.5 mg) were placed in a 10 ml syringe. To this powder was added 15 mg Sunitinib. In another syringe 0.6 ml sodium hydrogen phosphate buffer solution (0.01 molar, pH 6.9. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for elution study at 37° C. for about 6 months. 2m₁ of the buffer was removed each day and replaced with fresh 7.4 buffer. The amount of eluted product was analyzed by HPLC. The results indicated that about 4-6 micro grams of the sunitinib eluted each day for about 1.5 years. The study was stopped after 1.5 years

### Example 26: Preparation of Materials

A stirring 4,4′-Bis(mercaptomethyl)biphenyl solution (1.44 g) (Scheme 1) in ethanol/water (50/50, 5 ml) at ambient temperature was pH adjusted to 7.8 by adding glycylglycine solution. To this solution was added pentaerythritol tetraacrylate (1.05 gm.). The reactive mixture is poured into a desired surface mimicking wound and allowed to gel. It takes about 1-5 minutes to form the gel.

### Example 27: Preparation of Materials

A stirring 4,4′-bis(mercaptomethyl)biphenyl solution (1.44 g) (Scheme 1) in ethanol/water (50/50, 10 ml) at ambient temperature was pH adjusted to 7.8 by adding glycylglycine solution. To this solution was added 4-Arm PEG-Acrylate (2K MW), powder (6 gm.). The reactive mixture is poured into a desired surface mimicking wound and allowed to gel. It takes about 1-5 minutes to form the gel.

### Example 28: Preparation of Materials

A stirring 4,4′-bisphenol-4,4′-dithiol solution (1.31 g) (Scheme 2) in ethanol/water (50/50, 10 ml) at ambient temperature was pH adjusted to 7.8 by adding glycyl glycine solution.

To this solution was added 4-arm PEG-acrylate (2K MW), powder (6 gm.). The reactive mixture is poured into a desired surface mimicking wound and allowed to gel. It takes about 1-5 minutes to form the gel.

### Example 29: Preparation of Materials

A stirring glycidyl ether solution (1.44 g) (Scheme 3) in ethanol/water (50/50, 5 ml) at ambient temperature was pH adjusted to 7.8 by adding buffered saline solution. To this solution was added PEG amine and PEG amine ester. The reactive mixture is poured into a desired surface mimicking wound and allowed to gel. It takes about 1-5 minutes to form the gel.

### Example 30: Preparation of Materials

A stirring 4,4′-Bis(mercaptomelhyl)biphemi solution (1.44 g) (Scheme 1) in ethanol/water (50/50, 5 ml) at ambient temperature was pH adjusted to 7.8 by adding glycylglycine solution. To this solution was added pentaerythritol tetraacrylate (1.05 gm) and KB-R7885 (0.4 gm) or steroids (0.4 gm) etc. The reactive mixture is poured into a desired surface mimicking wound and allowed to gel. It takes about 1-5 minutes to form the gel.

### Example 31: Degradation of Polymer Materials

A mold piece manufactured per Example 22-26 is placed in a 10 ml buffered saline at 37° C. water bath. The degradation time is defined as the total dissolution of the gel. The degradation time is found to be 2-45 weeks depending on the formulations used.

### Example 32: Degradation Time and Drug Elution

A mold piece manufactured per Example 26 is placed in a 2 ml buffered saline at 37° C. water bath. The entire 2 ml saline solution is removed daily from the sample and a new 2 ml saline solution is added. The process is repeated daily until the device is completely dissolved. The saline sample with drug is analyzed by HPLC and GC to establish the elution rate.

### Example 33: Generation of Polymer Using a Photoinitiator

Starting materials according to any one of Schemes 1-7 or any of the preceding examples are provided. A photoinitiator (e.g., as described herein) is combined with the starting materials. UV or visible radiation is applied to initiate generation of a reactive species, which reactive species promotes the generation of a polymer according to the present disclosure.

### Example 34: Treatment of an Ophthalmic Condition

A polymer is prepared according to any one of Schemes 1-7 or any of the preceding examples. The polymer is combined with a therapeutic agent (e.g., an agent effective in treating an ophthalmic condition such as wet AMD, as described herein) to provide a therapeutic composition. The composition is provided in a syringe and is injected into the eye of a patient having an ophthalmic condition (e.g., wet AMD). The composition provides sustained release of the therapeutic agent over several weeks.

### Example 35: Treatment of an Ophthalmic Condition

Starting materials according to any one of Schemes 1-7 or any of the preceding examples are provided. The starting materials are combined with a therapeutic agent (e.g., an agent effective in treating an ophthalmic condition such as wet AMD, as described herein). A polymer is generated (e.g., as described herein) to provide a therapeutic composition. The composition is provided in a syringe and is injected into the eye of a patient having an ophthalmic condition (e.g., wet AMD). The composition provides sustained release of the therapeutic agent over several weeks.

### Example 36: Treatment of an Ophthalmic Condition Using an Implantable Device

A polymer is prepared according to any one of Schemes 1-7 or any of the preceding examples. The polymer is optionally combined with a therapeutic agent (e.g., an agent effective in treating an ophthalmic condition such as wet AMD, as described herein) to provide a composition (e.g., therapeutic composition). A drainage implant is coated with the composition. The implant (e.g., shunt) is implanted behind the upper or lower eyelid of an eye after administration of local anesthesia.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### API ELUTION STUDIES

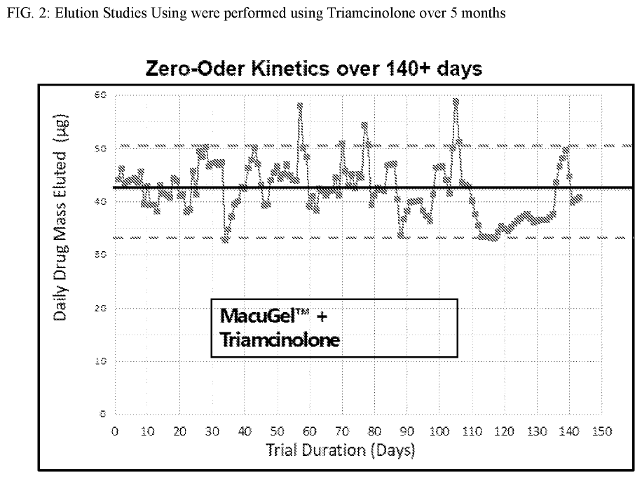

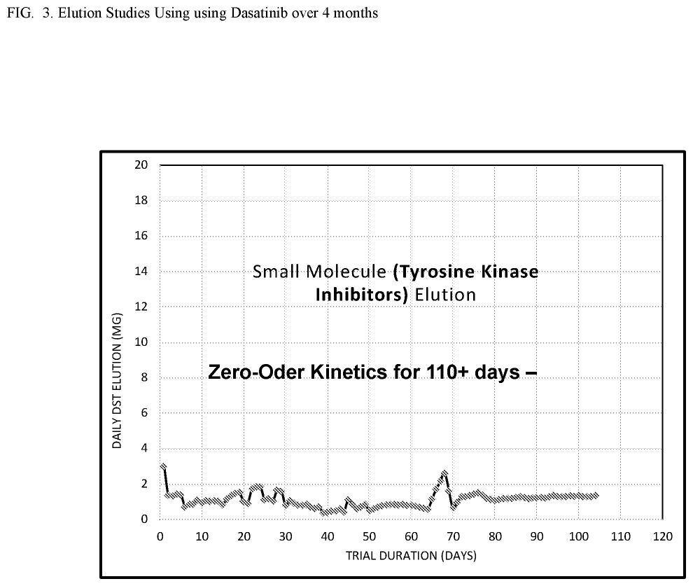

Selected examples of the elution studies using the MacuGel are shown in FIGS. 1-3. In each case the gel allows zero order kinetics over an extended period of time.

1. A composition comprising a biodegradable polymer, wherein said biodegradable polymer is generated by combining:
(a) at least one first compound comprising two or more thiol moieties and (i) two or more ethoxy moieties or (ii) two or more carboxyl moieties;
(b) at least one second compound comprising one or more acrylate moieties, and
(c) a therapeutic agent effective in the treatment of an ophthalmic condition.

2. The composition of claim 1, wherein said at least one first compound comprises three or more thiol moieties.

3. The composition of claim 1, wherein said at least one first compound comprises three or more carboxyl moieties.

4. The composition of claim 1, wherein said at least one first compound comprises three or more thiol moieties, wherein said thiol moieties are each represented by the formula: 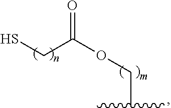
wherein n and m are for each thiol moiety are each independently selected from the group consisting of 1, 2, and 3.

5. The composition of claim 4, wherein said at least one first compound comprises at least six or more of said thiol moieties.

6. The composition of claim 5, wherein said at least one first compound comprises eight of said thiol moieties.

7. The composition of claim 1, wherein each n is 2 and each m is 1.

8. The composition of claim 4, wherein said at least one first compound is represented by the formula: 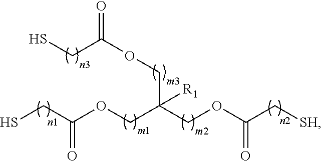
wherein R₁ is selected from the group consisting of H and C₁₋₆ alkyl;
n₁, n₂, and n₃ are each independently selected from the group consisting of 1, 2, and 3; and
m₁, m₂, and m₃ are each independently selected from the group consisting of 1, 2, and 3.

9. The composition of claim 8, wherein R₁ is C₁ alkyl.

10. The composition of claim 8, wherein n₁, n₂, and n₃ are each 2.

11. The composition of claim 8, wherein m₁, m₂, and m₃ are each 1.

12. The composition of claim 8, wherein said at least one first compound is represented by the formula:

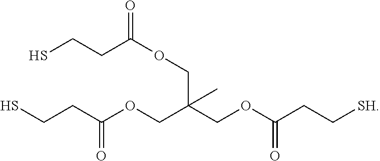 [SMILES: CC(COC(=O)CCS)(COC(=O)CCS)COC(=O)CCS]

13. The composition of claim 1, wherein said at least one first compound comprises three or more ethoxy moieties.

14. The composition of claim 13, wherein said at least one first compound comprises three or more thiol moieties, wherein said thiol moieties are each represented by the formula: 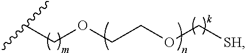
wherein k and m for each thiol moiety are each independently selected from the group consisting of 1, 2, and 3 and wherein n is selected from the group consisting of 1-500.

15. The composition of claim 14, wherein said at least one compound comprises at least six of said thiol moieties.

16. The composition of claim 15, wherein said at least one compound comprises eight of said thiol moieties.

17. The composition of claim 16, wherein each m is 1.

18. The composition of claim 16, wherein each k is 2.

19. The composition of claim 14, wherein said at least one first compound is represented by the formula: 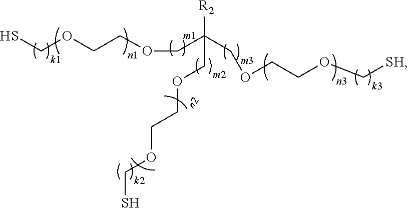
where R₂ is selected from H, C₁₋₆ alkyl, and 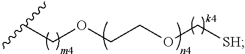
n₁, n₂, n₃, and n₄ (if present) are each independently selected from the group consisting of 1-500;
m₁, m₂, m₃, and m₄ (if present) are each independently selected from the group consisting of 1, 2, and 3; and
k₁, k2, k₃, and k₄ (if present) are each independently selected from the group consisting of 1, 2, and 3.

20. The composition of claim 19, wherein said at least one first compound is represented by the formula:

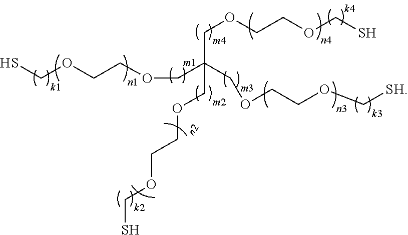

21-168. (canceled)
